(19) United States
(12) Patent Application Publication
(10) Pub. No.: US 2026/0201475 A1
(43) Pub. Date: Jul. 16, 2026

(54) METHODS FOR DETECTING AND TREATING OVARIAN CANCER

(71) Applicant: UNIVERSITY OF IOWA RESEARCH FOUNDATION, Iowa City, IA (US)

(72) Inventors: Jesus Gonzalez Bosquet, Iowa City, IA (US); Nicholas D. Cardillo, Iowa City, IA (US); Eric Devor, Iowa City, IA (US); Brian J. Smith, Iowa City, IA (US); Michael J. Goodheart, Iowa City, IA (US)

(73) Assignee: UNIVERSITY OF IOWA RESEARCH FOUNDATION, Iowa City, IA (US)

(21) Appl. No.: 19/133,108

(22) PCT Filed: Nov. 27, 2023
(86) PCT No.: PCT/US2023/081148

§ 371 Date: May 27, 2025

Related U.S. Application Data
(60) Provisional application No. 63/428,346, filed on Nov. 28, 2022.

(51) Int. Cl. C12Q 1/6886

(52) U.S. Cl. CPC C12Q 1/6886; C12Q 2600/118; C12Q 2600/156

(57) ABSTRACT

In certain embodiments, the present invention provides a panel of probes associated with ovarian cancer that hybridize to nucleic acid and kits that comprise a panel of probes associated with ovarian cancer. In certain embodiments, the present invention provides a method of detecting the presence of biomarkers associated with an increased risk of ovarian cancer in a human subject. In certain embodiments, the present invention provides a method of treating a human subject for ovarian cancer.

### CROSS-REFERENCE TO RELATED APPLICATIONS

This application claims priority to U.S. Provisional Application No. 63/428,346 that was filed on Nov. 28, 2022. The entire content of the applications referenced above is hereby incorporated by reference herein.

### GOVERNMENT FUNDING

This invention was made with government support under CA099908 awarded by the National Institutes of Health, and under OC190352 awarded by the Department of Defense. The government has certain rights in the invention.

### BACKGROUND

Molecular profiling of tumors obtained from individual patients improves the selection of personalized cancer treatment therapies, patient responses, detection of drug resistance, and monitoring of tumor relapse. Profiling tumors generally involves obtaining resected tumor samples by invasive surgeries. The limitations to such invasive procedures include difficulty in acquiring tumor samples for both tumor quantity and quality. Another drawback is that acquiring biopsy samples by invasive methods throughout treatment to monitor tumor response and relapse pose major challenges in tumor profiling. A further limitation to invasive sampling methods is the heterogeneity of resected tumor samples as a whole. Further, in the case of metastasis, where tumors have spread and constantly evolve both spatially and temporally in response to treatment over time, multiple biopsies may be required. These challenges make it difficult to obtain a holistic image of a tumor.

Recently, new non-invasive techniques are being developed to address these limitations, such as liquid biopsy (LB). Liquid biopsies consist of isolating tumor-derived entities like circulating tumor cells, circulating tumor DNA, tumor extracellular vesicles, etc., present in the body fluids of patients with cancer, followed by an analysis of genomic and proteomic data contained within them. Liquid biopsies methods permit continuous monitoring by repeated sampling. Further, LB provides enhanced sensitivity in diagnosis and ease of repeated sampling throughout treatment much more conveniently and non-invasively.

Various groups have attempted to increase the accuracy of pre-operative diagnosis of pelvic masses. Traditionally, the methods involved tumor markers, such as CA-125, HE-4, among others with or without the addition of ultrasound imaging characteristics and patient menopausal status. These methods offer sensitivities and specificities in the 70-80% range. More recently, groups have begun using machine learning and genomic information to create models that would better identify these models. To this point, however, they have not yet improved upon the tumor marker model. Accordingly, a minimally invasive model is needed that increases the accuracy of pre-operative diagnosis of pelvic masses.

### SUMMARY

One aspect provides a panel of probes associated with ovarian cancer that hybridize to nucleic acid, the panel comprising at least 18 probes selected from the group consisting of the probes listed in FIG. 3B.

In certain aspects, the panel comprises at least 40 probes.

In certain aspects, the panel comprises at least 49 probes.

In certain aspects, the probes are specific for single nucleotide variants (SNVs).

In certain aspects, the probes are specific for copy number variants (CNVs).

In certain aspects, the probes are specific for structural variants (SVs).

In certain aspects, each probe comprises a unique label.

One aspect provides a kit comprising a panel of probes associated with ovarian cancer that hybridize to nucleic acid, the panel comprising at least 18 probes selected from the group consisting of the probes listed in FIG. 3B, and instructions for use in analyzing a biological sample.

In certain aspects, the biological sample is a liquid biopsy.

In certain aspects, the liquid biopsy is blood or a blood product.

One aspect provides a method of detecting the presence of biomarkers associated with an increased risk of ovarian cancer in a human subject, comprising:
(i) contacting DNA from a biological sample containing cells from the subject with a panel of probes associated with ovarian cancer that hybridize to nucleic acid, the panel comprising at least 18 probes selected from the group consisting of the probes listed in FIG. 3B, wherein the panel comprises probes specific for single nucleotide variants (SNVs) to form hybridized target sequences;
(ii) detecting the hybridized target sequences;
(iii) determining the number of hybridized target sequences detected; and
(iv) indicating that the human subject has an increased risk of ovarian cancer if more than 18 SNVs are present.

In certain aspects, the biological sample is a liquid biopsy.

In certain aspects, the liquid biopsy is blood or a blood product.

In certain aspects, the biological sample is subdivided into individual subsamples, and a different single probe is applied to each subsample.

One aspect provides a method of treating a human subject for ovarian cancer, comprising:
(i) collecting a liquid biopsy from the subject, and extracting ctDNA from the liquid biopsy;
(ii) analyzing the presence or absence of ctDNA listing in a panel of loci selected from the group consisting of the probes listed in FIG. 3B;
(iii) determining the number of matching loci are present;
(iv) indicating that the human subject has an increased risk of ovarian cancer if at least 18 loci are present; and
(v) administering an appropriate treatment to the patient.

In certain aspects, the panel comprises at least 40 probes.

In certain aspects, the panel comprises at least 49 probes.

In certain aspects, the treatment comprises assessment of fallopian tubes and ovaries with imaging techniques or removal of tubes and ovaries. In certain aspects, the imaging techniques are CT scan, MRI and/or ultrasound.

One aspect provides a method of determining an increased risk or presence of ovarian cancer in a human subject comprising:
(i) collecting a liquid biopsy from the subject, and extracting ctDNA from the liquid biopsy;
(ii) analyzing the presence or absence of ctDNA listing in a panel of loci selected from the group consisting of the probes listed in FIG. 3B;
(iii) determining the number of matching loci are present; and
(iv) indicating that the human subject has an increased risk of ovarian cancer if at least 18 loci are present.

In certain aspects, the panel comprises at least 40 probes.

In certain aspects, the panel comprises at least 49 probes.

### BRIEF DESCRIPTION OF DRAWINGS

FIG. 1: Variation analysis with VEP. Of the initial approximately 31 million SNVs found in all HGSC samples, 11.3 million were in unique loci. Over 242,000 of them were already present in normal controls from the gnomAD database, with no differences in allele frequencies, p-value<0.05, from more than 125,000 individuals. These were subtracted from the analysis. The resultant SNVs were assessed for their association with HGSC with logistic regression. Unrelated controls consisted of 14 DNA samples from the distal part of the Fallopian tube (fimbria) from patients with no disease and no family history of ovarian cancer. This resulted in 16,631 significant SNVs, associated with HGSC, at a p-value <10⁻³ to account for multiple comparisons.

Variation analysis was then performed with superFreq. Of the initial approximately 220 thousand SNVs found in all HGSC samples after quality filters, 118,839 were in unique loci. 100,164 of them were already present in normal controls from the gnomAD database or in the previous analysis with VEP. These were subtracted from the analysis. The resultant 18,675 SNVs were assessed for their association with HGSC with logistic regression, as in VEP analysis. It resulted in 5 significant SNVs, associated with HGSC, at a p-value 0.05.

FIGS. 2A and 2B: Variation analysis with VEP and superFreq. FIG. 2A. Manhattan plot representation of 16,631 significant SNVs associated with HGSC, at a p-value <10⁻³, after VEP SNV determination and logistic regression analyses comparing HGSC and normal tube. The x axis represents location of SNV within chromosomes; the y axis represents the ORs of the association with HGSC; the red line designates p=10⁻³ FIG. 2B. Representation of 5 significant SNVs (out of 18,675) associated with HGSC, at a p-value <0.05, after superFreq SNV determination and logistic regression analyses comparing HGSC and normal tube. These SNVs were not identified within the VEP analysis. The x axis represent location of SNV within chromosomes; the y axis represents the log transformation of the p-value.

FIGS. 3A and 3B: Lasso multivariate regression analysis of 16,636 significant SNVs in the univariate analysis. The model had a performance, measured in AUC of 1.0, when it included 49 SNVs (FIG. 3A). Details of these SNVs are represented in the table (FIG. 3B). Only one locus conferred substantial protection to HGSC: chr1_KI270706v1_random:30898, (allele G). The other loci's risks were insignificant. The majority of these SNVs have been already described (RS column).

FIGS. 4A-4C: Variation analysis with VEP and superFreq in RNA-seq experiments FIG. 4A. 6,296 SNVs were also present in RNA-seq VEP analysis (out of 16,631 significant SNVs, associated with HGSC). In the univariate analysis, 532 SNVs were associated with HGSC (p-value <0.05). FIG. 4B. Multivariate logistic regression with all 532 significant in the univariate analysis. Three loci were independently associated with HGSC (p<0.05). These 3 loci represent 3 SNPs already described (column RS), and all of them conferred protection against HGSC. FIG. 4C. Lasso prediction model independent of DNA model: We introduced all 532 SNVs that were significant in the univariate analysis in a lasso prediction model. The model selected 4 loci: chr1:118080760, chr1:114091254, chr3:57511228, and chr6:49709492 that predict HGSC with an AUC of 99%.

FIGS. 5A-5C: Gene copy number (CNV) analysis: there were 558 genes with differential copy number out of all 23,443 genes, at a p-value <10⁻³ (to adjust for multiple comparisons). FIG. 5A. Manhattan plot representations of all 558 CNV. The red lines represent ×2 copies (or more than diploid) and 0.5 copies (or less than heterozygous). FIG. 5B. Logistic regression analysis to determine independently CNV associated with HGSC. FIG. 5C. ROC curve of the prediction model with 11 transcripts with CNV: AUC of 87%, 95% CI:72%,100%). All of them increase the risk with relative moderate OR. Below the ROC is the table with transcript name and location.

FIGS. 6A-6C: Comparison of structural variation (SV) between HGSC samples and tubal controls. Analysis performed with MINTIE: an integrated pipeline for RNA-seq data that takes a reference-free approach, combining de novo assembly of transcripts with differential expression analysis to identify up-regulated novel variants in a case-control setting. (FIG. 6A) All SV for each individual case (122), each one compared to controls (12). (FIG. 6B) Significant SV are represented in a Manhattan plot, with a p-value cut-off of 10⁻⁵, to adjust for multiple comparisons (FDR). (FIG. 6C) Then all significant SV at the sample level were introduced in logistic models to compare all cases vs all controls.

FIGS. 7A-7D: Validation of WES DNA SNV prediction model of HGSC performed in RNA-seq samples with machine learning analytical platform. FIG. 7A. Model with all SNV associated with HGSC (N=16,631). The superior panel shows the confusion matrix representing the observed versus the predicted values. The inferior panel is an ROC graphic: true positives in the x axis, false positives in the y axis, and AUC results. Train B: results of baseline training; Test B: results of baseline testing. FIG. 7B. Model resulting from the multivariate analysis with lasso (N=49). Superior and inferior panels are as before. FIG. 7C. Model with UI RNA-seq SNVs (N=20). Superior panel is as before. Inferior panel represents the ROC graphic including models accounting for weights of the outcome: 1) Train W: results of weighted model training; Test W: results of weighted model testing; 2) Train R: results of unbalanced (or re-sampling) model training; Test R: results of re-sampling model testing. FIG. 7D. Model with TCGA RNA-seq SNVs (N=18). Superior panel is as before. Inferior panel represents the ROC graphic including models accounting for weights of the outcome: Train R: results of unbalanced (or re-sampling) model training; Test R: results of re-sampling model testing.

FIGS. 8A-8E: Multivariate analysis of all cases vs all controls. FIG. 8A. Multivariate analysis with all 6,003 significant SV in the univariate analysis. FIG. 8B. Multivariate analysis with only novel exon (NE) significant SV in the univariate analysis. FIG. 8C. Multivariate analysis with only retained intron (RI) significant SV in the univariate analysis. FIG. 8D. Multivariate analysis with only partial novel junction (PNJ) significant SV in the univariate analysis. FIG. 8E. Multivariate analysis with only unknown (UN) significant SV in the univariate analysis. SV: structural variant. NE: novel exon—an aligned block that does not cross any existing exons. May be intronic or intergenic. RI: retained intron—a contig block that spans a whole intron. PNJ: partial novel junction—spliced contig where one junction matches a known boundary (the other side is unknown). UN: unknown—by default, all soft-clipped contigs are classified as unknown at the moment. * Significant at a p-value <0.05.

FIGS. 9A-9B: Multivariate Lasso prediction model of HGSC with SVs. 22 SV were initially significant in the ANOVA univariate analysis with cross-validation out of the total 32,156 SVs. FIG. 9A. The lasso multivariate prediction analysis identified 17 SV that predicted HGSC with an AUC of 73%. FIG. 9B. Prediction model with performance by AUC of 0.73 (95% CI: 69%-77%).

### DETAILED DESCRIPTION

### Definitions

Unless otherwise indicated, the practice of the method and system disclosed herein involves conventional techniques and apparatus commonly used in molecular biology, microbiology, protein purification, protein engineering, protein and DNA sequencing, and recombinant DNA fields, which are within the skill of the art. Such techniques and apparatus are known to those of skill in the art and are described in numerous texts and reference works (See e.g., Sambrook et al., “Molecular Cloning: A Laboratory Manual,” Third Edition (Cold Spring Harbor), [2001]); and Ausubel et al., “Current Protocols in Molecular Biology” [1987]).

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the Specification as a whole. It is to be understood that this disclosure is not limited to the particular methodology, protocols, and reagents described, as these may vary, depending upon the context they are used by those of skill in the art. As used herein, the singular terms “a,” “an,” and “the” include the plural reference unless the context clearly indicates otherwise.

Unless otherwise indicated, nucleic acids are written left to right in 5′ to 3′ orientation and amino acid sequences are written left to right in amino to carboxy orientation, respectively.

A sequence of interest as used herein indicates a nucleic acid sequence in a genome of an organism such as a human. In some implementations, the sequence of interest is a gene, a SNP, an exon, a regulatory sequence of a gene, etc. In some implementations, the sequence of interest is a chromosome or a sub-chromosomal region.

A variant of interest is particular variant of a genetic sequence that is to be measured, qualified, quantified, or detected. In some implementations, a variant of interest is a variant known or suspected to be associated with a condition, such as a cancer, a tumor, or a genetic disorder.

A gene is a locus (or region) of DNA which is made up of nucleotides and is the molecular unit of heredity.

Genes can acquire mutations in their sequence, leading to different variants, known as alleles, in the population. These alleles encode slightly different versions of a protein, which cause different phenotype traits.

Allele frequency or gene frequency, is the frequency of an allele of a gene (or a variant of the gene) relative to other alleles of the gene, which can be expressed as a fraction or percentage. An allele frequency is often associated with a particular genomic locus, because a gene is often located at with one or more locus. However, an allele frequency as used herein can also be associated with a size-based bin of DNA fragments. In this sense, DNA fragments such as cfDNA containing an allele are assigned to different size-based bins. The frequency of the allele in a size-based bin relative to the frequency of other alleles is an allele frequency. In some implementations, the frequency of an allele or a variant is a proportion of reads supporting the variant calls out of all reads in multiple bins, such as a prioritized set of bins.

The term “parameter” herein refers to a numerical value that characterizes a property of a system such as a physical feature whose value or other characteristic has an impact on a relevant condition such as a sample or DNA fragments having a simple nucleotide variant or a copy number variant. In some cases, the term parameter is used with reference to a variable that affects the output of a mathematical relation or model, which variable may be an independent variable (i.e., an input to the model) or an intermediate variable based on one or more independent variables. Depending on the scope of a model, an output of one model may become an input of another model, thereby becoming a parameter to the other model.

The term “fragment size parameter” refers to a parameter that relates to the size or length of a fragment or a collection of fragments such nucleic acid fragments; e.g., a cfDNA fragments obtained from a bodily fluid. A fragment size or size range may be a characteristic of an aberrant genome or a portion thereof when the genome produces nucleic acid fragments having a higher concentration of the size or size range relative to nucleic acid fragments from another genome or another portion of the same genome. Various implementations disclosed herein provide methods to combine size information with sequence information to determine simple nucleotide variants. Additionally, the abundance of sequences can also be combined with size information to determine a structural variation or a copy number variation. Various implementations combine fragment size information and sequence information in innovative ways that are more efficient than simple additions or alternative selections of the two kinds of information, thereby providing improved performance over conventional assays for detecting cancer variants having low variant frequency.

“Single Nucleotide Variants” or “SNVs” are genetic variants that differ from a reference sequence by one nucleotide in a relatively short genetic sequence. SNVs are distinct from structural variant and copy number variant (CNVs) in that structural variants (SVs) include chromosomal structural rearrangements such as large indels, duplications, inversions, and transversions, and copy number variants include abnormal copy numbers of normally diploid regions of the genome. Some SNVs known to or suspected to be associated with tumors, also referred to as tumor SNVs, are targeted for analysis in various implementations.

The term “potentially variant-containing fragment” is used herein to refer to fragments that are identified as cfDNA fragments that are suspected of harboring a sequence mutation corresponding to a cancer variant. In various implementations, a cfDNA fragment is identified as a potentially variant-containing fragment when it is determined that the fragment provides a sequence read that includes a sequence of a known cancer variant and that the sequence read's genomic coordinate matches that of the cancer variant. Because sequencing and other processing sometimes introduces errors, there is uncertainty that a fragment sequence showing a cancer mutation actually corresponds to a fragment originating from a cancer cell. There is some chance that a cancer variant-containing sequence read from a fragment is in fact due to sequencing errors instead of an actual somatic mutation.

The term “copy number variation” or “CNV” herein refers to variation in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A “copy number variant” refers to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations include deletions, including microdeletions, insertions, including microinsertions, duplications, multiplications, and translocations. CNVs encompass chromosomal aneuploidies and partial aneuploidies.

The term “aneuploidy” herein refers to an imbalance of genetic material caused by a loss or gain of a whole chromosome, or part of a chromosome.

The term “plurality” refers to more than one element. For example, the term is used herein in reference to a number of nucleic acid molecules or sequence tags that are sufficient to identify significant differences in SNVs, CNVs or SVs in test samples and qualified samples using the methods disclosed herein. In some embodiments, at least about 3×10⁶ sequence tags of between about 20 and 40 bp are obtained for each test sample. In some embodiments, each test sample provides data for at least about 5×10⁶, 8×10⁶, 10×10⁶, 15×10⁶, 20×10⁶, 30×10⁶, 40×10⁶, or 50×10⁶ sequence tags, each sequence tag comprising between about 20 and 40 bp.

The term “paired end reads” refers to reads from paired end sequencing that obtains one read from each end of a nucleic acid fragment. Paired end sequencing may involve fragmenting strands of polynucleotides into short sequences called inserts. Fragmentation is optional or unnecessary for relatively short polynucleotides such as cell free DNA molecules.

The terms “polynucleotide,” “nucleic acid” and “nucleic acid molecules” are used interchangeably and refer to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The nucleotides include sequences of any form of nucleic acid, including, but not limited to RNA and DNA molecules such as cfDNA molecules. The term “polynucleotide” includes, without limitation, single- and double-stranded polynucleotide.

The term “test sample” herein refers to a sample, typically derived from a biological fluid, cell, tissue, organ, or organism, comprising a nucleic acid or a mixture of nucleic acids comprising at least one nucleic acid sequence that is to be screened for SNVs, CNVs or SVs. In certain embodiments the sample comprises at least one nucleic acid sequence whose copy number is suspected of having undergone variation. Such samples include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like. Although the sample is often taken from a human subject (e.g., patient), the assays can be used to SNVs, CNVs or SVs in samples from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “training set” herein refers to a set of training samples that can comprise affected and/or unaffected samples and are used to develop a model for analyzing test samples. In some embodiments, the training set includes unaffected samples. In these embodiments, thresholds for detecting SNVs, CNVs or SVs are established using training sets of samples that are unaffected for the SNVs, CNVs or SVs of interest. The unaffected samples in a training set may be used as the qualified samples to identify normalizing sequences, e.g., normalizing chromosomes, and the chromosome doses of unaffected samples are used to set the thresholds for each of the sequences, e.g., chromosomes, of interest. In some embodiments, the training set includes affected samples. The affected samples in a training set can be used to verify that affected test samples can be easily differentiated from unaffected samples.

A training set is also a statistical sample in a population of interest, which statistical sample is not to be confused with a biological sample. A statistical sample often comprises multiple individuals, data of which individuals are used to determine one or more quantitative values of interest generalizable to the population. The statistical sample is a subset of individuals in the population of interest. The individuals may be persons, animals, tissues, cells, other biological samples (i.e., a statistical sample may include multiple biological samples), and other individual entities providing data points for statistical analysis.

Usually, a training set is used in conjunction with a validation set. The term “validation set” is used to refer to a set of individuals in a statistical sample, data of which individuals are used to validate or evaluate the quantitative values of interest determined using a training set. In some embodiments, for instance, a training set provides data for calculating a mask for a reference sequence, while a validation set provides data to evaluate the validity or effectiveness of the mask.

The term “sequence of interest” or “nucleic acid sequence of interest” herein refers to a nucleic acid sequence that is associated with a difference in sequence representation between healthy and diseased individuals. A sequence of interest can be a sequence on a chromosome that is misrepresented, i.e., over- or under-represented, in a disease or genetic condition. A sequence of interest may be a portion of a chromosome, i.e., chromosome segment, or a whole chromosome. For example, a sequence of interest can be a chromosome that is over-represented in an aneuploidy condition, or a gene encoding a tumor-suppressor that is under-represented in a cancer. Sequences of interest include sequences that are over- or under-represented in the total population, or a subpopulation of cells of a subject. A “qualified sequence of interest” is a sequence of interest in a qualified sample. A “test sequence of interest” is a sequence of interest in a test sample.

The term “coverage” refers to the abundance of sequence tags mapped to a defined sequence. Coverage can be quantitatively indicated by sequence tag density (or count of sequence tags), sequence tag density ratio, normalized coverage amount, adjusted coverage values, etc.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The terms “threshold value” and “qualified threshold value” herein refer to any number that is used as a cutoff to characterize a sample such as a test sample containing a nucleic acid from an organism suspected of having a medical condition. The threshold may be compared to a parameter value to determine whether a sample giving rise to such parameter value suggests that the organism has the medical condition. In certain embodiments, a qualified threshold value is calculated using a qualifying data set and serves as a limit of diagnosis of a SNVs, CNVs or SVs. If a threshold is exceeded by results obtained from methods disclosed herein, a subject can be diagnosed with a SNVs, CNVs or SVs. Appropriate threshold values for the methods described herein can be identified by analyzing normalized values (e.g. chromosome doses, NCVs or NSVs) calculated for a training set of samples. Threshold values can be identified using qualified (i.e., unaffected) samples in a training set which comprises both qualified (i.e., unaffected) samples and affected samples. The samples in the training set known to have chromosomal aneuploidies (i.e., the affected samples) can be used to confirm that the chosen thresholds are useful in differentiating affected from unaffected samples in a test set (see the Examples herein). The choice of a threshold is dependent on the level of confidence that the user wishes to have to make the classification. In some embodiments, the training set used to identify appropriate threshold values comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, or more qualified samples. It may be advantageous to use larger sets of qualified samples to improve the diagnostic utility of the threshold values.

The term “read” refers to a sequence obtained from a portion of a nucleic acid sample. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

The term “genomic read” is used in reference to a read of any segments in the entire genome of an individual.

The term “sequence tag” is herein used interchangeably with the term “mapped sequence tag” to refer to a sequence read that has been specifically assigned, i.e., mapped, to a larger sequence, e.g., a reference genome, by alignment. Mapped sequence tags are uniquely mapped to a reference genome, i.e., they are assigned to a single location to the reference genome. Unless otherwise specified, tags that map to the same sequence on a reference sequence are counted once. Tags may be provided as data structures or other assemblages of data. In certain embodiments, a tag contains a read sequence and associated information for that read such as the location of the sequence in the genome, e.g., the position on a chromosome. In certain embodiments, the location is specified for a positive strand orientation. A tag may be defined to allow a limited amount of mismatch in aligning to a reference genome. In some embodiments, tags that can be mapped to more than one location on a reference genome, i.e., tags that do not map uniquely, may not be included in the analysis.

As used herein, the terms “aligned,” “alignment,” or “aligning” refer to the process of comparing a read or tag to a reference sequence and thereby determining whether the reference sequence contains the read sequence. If the reference sequence contains the read, the read may be mapped to the reference sequence or, in certain embodiments, to a particular location in the reference sequence. In some cases, alignment simply tells whether or not a read is a member of a particular reference sequence (i.e., whether the read is present or absent in the reference sequence). For example, the alignment of a read to the reference sequence for human chromosome 13 will tell whether the read is present in the reference sequence for chromosome 13. A tool that provides this information may be called a set membership tester. In some cases, an alignment additionally indicates a location in the reference sequence where the read or tag maps to. For example, if the reference sequence is the whole human genome sequence, an alignment may indicate that a read is present on chromosome 13, and may further indicate that the read is on a particular strand and/or site of chromosome 13.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. One example of an algorithm from aligning sequences is the Efficient Local Alignment of Nucleotide Data (ELAND) computer program distributed as part of the Illumina Genomics Analysis pipeline. Alternatively, a Bloom filter or similar set membership tester may be employed to align reads to reference genomes. See U.S. Patent Application No. 61/552,374 filed Oct. 27, 2011, which is incorporated herein by reference in its entirety. The matching of a sequence read in aligning can be a 100% sequence match or less than 100% (non-perfect match).

The term “mapping” used herein refers to specifically assigning a sequence read to a larger sequence, e.g., a reference genome, by alignment.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. A “genome” refers to the complete genetic information of an organism or virus, expressed in nucleic acid sequences.

In various embodiments, the reference sequence is significantly larger than the reads that are aligned to it. For example, it may be at least about 100 times larger, or at least about 1000 times larger, or at least about 10,000 times larger, or at least about 10⁵ times larger, or at least about 10⁶ times larger, or at least about 10⁷ times larger.

In one example, the reference sequence is that of a full-length human genome. Such sequences may be referred to as genomic reference sequences. In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), etc., of any species.

In various embodiments, the reference sequence is a consensus sequence or other combination derived from multiple individuals. However, in certain applications, the reference sequence may be taken from a particular individual.

The term “clinically-relevant sequence” herein refers to a nucleic acid sequence that is known or is suspected to be associated or implicated with a genetic or disease condition. Determining the absence or presence of a clinically-relevant sequence can be useful in determining a diagnosis or confirming a diagnosis of a medical condition, or providing a prognosis for the development of a disease.

The term “derived” when used in the context of a nucleic acid or a mixture of nucleic acids, herein refers to the means whereby the nucleic acid(s) are obtained from the source from which they originate. For example, in one embodiment, a mixture of nucleic acids that is derived from two different genomes means that the nucleic acids, e.g., cfDNA, were naturally released by cells through naturally occurring processes such as necrosis or apoptosis. In another embodiment, a mixture of nucleic acids that is derived from two different genomes means that the nucleic acids were extracted from two different types of cells from a subject.

The term “based on” when used in the context of obtaining a specific quantitative value, herein refers to using another quantity as input to calculate the specific quantitative value as an output.

The term “biological fluid” herein refers to a liquid taken from a biological source and includes, for example, blood, serum, plasma, sputum, lavage fluid, cerebrospinal fluid, urine, semen, sweat, tears, saliva, and the like. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

As used herein, the term “corresponding to” sometimes refers to a nucleic acid sequence, e.g., a gene or a chromosome, that is present in the genome of different subjects, and which does not necessarily have the same sequence in all genomes, but serves to provide the identity rather than the genetic information of a sequence of interest, e.g., a gene or chromosome.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

As used herein, the term “polynucleotide length” refers to the absolute number of nucleotides in a sequence or in a region of a reference genome. The term “chromosome length” refers to the known length of the chromosome given in base pairs, e.g., provided in the NCBI36/hg18 assembly of the human chromosome found at |genome|.|ucsc|.|edu/cgi-bin/hgTracks?hgsid=167155613&chromInfoPage=on the World Wide Web.

The term “subject” herein refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

The term “condition” herein refers to “medical condition” as a broad term that includes all diseases and disorders, but can include injuries and normal health situations, such as pregnancy, that might affect a person's health, benefit from medical assistance, or have implications for medical treatments.

The term “sensitivity” as used herein refers to the probability that a test result will be positive when the condition of interest is present. It may be calculated as the number of true positives divided by the sum of true positives and false negatives.

The term “specificity” as used herein refers to the probability that a test result will be negative when the condition of interest is absent. It may be calculated as the number of true negatives divided by the sum of true negatives and false positives.

The term “enrich” herein refers to the process of amplifying polymorphic target nucleic acids contained in a portion of a biological sample and combining the amplified product with the remainder of the biological sample from which the portion was removed. For example, the remainder of the biological sample can be the original biological sample.

The term “primer,” as used herein refers to an isolated oligonucleotide that is capable of acting as a point of initiation of synthesis when placed under conditions inductive to synthesis of an extension product (e.g., the conditions include nucleotides, an inducing agent such as DNA polymerase, and a suitable temperature and pH). The primer is preferably single stranded for maximum efficiency in amplification but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design.

### Sequencing Methods

As indicated above, the prepared samples (e.g., Sequencing Libraries) are sequenced as part of the procedure for identifying SNVs, CNVs or SVs. Any of a number of sequencing technologies can be utilized.

Some sequencing technologies are available commercially, such as the sequencing-by-hybridization platform from Affymetrix Inc. (Sunnyvale, Calif) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.), as described below. In addition to the single molecule sequencing performed using sequencing-by-synthesis of Helicos Biosciences, other single molecule sequencing technologies include, but are not limited to, the SMRT™ technology of Pacific Biosciences, the ION TORREN™ technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM). Illustrative sequencing technologies are described in greater detail below.

In one illustrative, but non-limiting, embodiment, the methods described herein comprise obtaining sequence information for the nucleic acids in a test sample (e.g., cfDNA or cellular DNA in a subject being screened for a cancer) using Illumina's sequencing-by-synthesis and reversible terminator-based sequencing chemistry. Template DNA can be genomic DNA, e.g., cellular DNA or cfDNA. In some embodiments, genomic DNA from isolated cells is used as the template, and it is fragmented into lengths of several hundred base pairs. In other embodiments, cfDNA is used as the template, and fragmentation is not required as cfDNA exists as short fragments. Circulating tumor DNA also exist in short fragments, with a size distribution peaking at about 150-170 bp. Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchor oligos (not to be confused with the anchor/anchored reads in the analysis of repeat expansion). Under limiting-dilution conditions, adapter-modified, single-stranded template DNA is added to the flow cell and immobilized by hybridization to the anchor oligos. Attached DNA fragments are extended and bridge amplified to create an ultra-high density sequencing flow cell with hundreds of millions of clusters, each containing about 1,000 copies of the same template. In one embodiment, the randomly fragmented genomic DNA is amplified using PCR before it is subjected to cluster amplification. Alternatively, an amplification-free (e.g., PCR free) genomic library preparation is used, and the randomly fragmented genomic DNA is enriched using the cluster amplification alone. The templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about tens to a few hundred base pairs are aligned against a reference genome and unique mapping of the short sequence reads to the reference genome are identified using specially developed data analysis pipeline software. After completion of the first read, the templates can be regenerated in situ to enable a second read from the opposite end of the fragments. Thus, either single-end or paired end sequencing of the DNA fragments can be used.

Various embodiments of the disclosure may use sequencing by synthesis that allows paired end sequencing. In some embodiments, the sequencing by synthesis platform by Illumina involves clustering fragments. Clustering is a process in which each fragment molecule is isothermally amplified. In some embodiments, as the example described here, the fragment has two different adaptors attached to the two ends of the fragment, the adaptors allowing the fragment to hybridize with the two different oligos on the surface of a flow cell lane. The fragment further includes or is connected to two index sequences at two ends of the fragment, which index sequences provide labels to identify different samples in multiplex sequencing. In some sequencing platforms, a fragment to be sequenced is also referred to as an insert.

In some implementation, a flow cell for clustering in the Illumina platform is a glass slide with lanes. Each lane is a glass channel coated with a lawn of two types of oligos. Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification, a strand folds over, and a second adapter region on a second end of the strand hybridizes with the second type of oligos on the flow cell surface. A polymerase generates a complimentary strand, forming a double-stranded bridge molecule. This double-stranded molecule is denatured resulting in two single-stranded molecules tethered to the flow cell through two different oligos. The process is then repeated over and over, and occurs simultaneously for millions of clusters resulting in clonal amplification of all the fragments. After bridge amplification, the reverse strands are cleaved and washed off, leaving only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters are sequenced in a massively parallel manner. At the completion of the first read, the read product is washed away.

In the next step of protocols involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flow cell. An index 2 sequence is read in the same manner as index 1. Then an index 2 read product is washed off at the completion of the step.

After reading two indices, read 2 initiates by using polymerases to extend the second flow cell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences. These contiguous sequences are aligned to the reference genome for variant identification.

The sequencing by synthesis example described above involves paired end reads, which is used in many of the embodiments of the disclosed methods. Paired end sequencing involves two reads from the two ends of a fragment. When a pair of reads are mapped to a reference sequence, the base-pair distance between the two reads can be determined, which distance can then be used to determine the length of the fragments from which the reads were obtained. In some instances, a fragment straddling two bins would have one of its pair-end read aligned to one bin, and another to an adjacent bin. This gets rarer as the bins get longer or the reads get shorter. Various methods may be used to account for the bin-membership of these fragments. For instance, they can be omitted in determining fragment size frequency of a bin; they can be counted for both of the adjacent bins; they can be assigned to the bin that encompasses the larger number of base pairs of the two bins; or they can be assigned to both bins with a weight related to portion of base pairs in each bin.

Paired end reads may use insert of different length (i.e., different fragment size to be sequenced). As the default meaning in this disclosure, paired end reads are used to refer to reads obtained from various insert lengths. In some instances, to distinguish short-insert paired end reads from long-inserts paired end reads, the latter is also referred to as mate pair reads. In some embodiments involving mate pair reads, two biotin junction adaptors first are attached to two ends of a relatively long insert (e.g., several kb). The biotin junction adaptors then link the two ends of the insert to form a circularized molecule. A sub-fragment encompassing the biotin junction adaptors can then be obtained by further fragmenting the circularized molecule. The sub-fragment including the two ends of the original fragment in opposite sequence order can then be sequenced by the same procedure as for short-insert paired end sequencing described above.

After sequencing of DNA fragments, sequence reads of predetermined length, e.g., 100 bp, are mapped or aligned to a known reference genome. The mapped or aligned reads and their corresponding locations on the reference sequence are also referred to as tags. In one embodiment, the reference genome sequence is the NCBI36/hg18 sequence, which is available on the world wide web at genome dot ucsc dot edu/cgi-bin/hgGateway?org=Human&db=hg18&hgsid=166260105). Alternatively, the reference genome sequence is the GRCh37/hg19, which is available on the world wide web at genome dot ucsc dot edu/cgi-bin/hgGateway. Other sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al., Genome Biology 10:R25.1-R25.10 [2009]), or ELAND (Illumina, Inc., San Diego, Calif., USA). In one embodiment, one end of the clonally expanded copies of the plasma cfDNA molecules is sequenced and processed by bioinformatics alignment analysis for the Illumina Genome Analyzer, which uses the Efficient Large-Scale Alignment of Nucleotide Databases (ELAND) software.

In one illustrative, but non-limiting, embodiment, the methods described herein comprise obtaining sequence information for the nucleic acids in a test sample, e.g., cfDNA or cellular DNA in a subject being screened for a cancer using single molecule sequencing technology of the Helicos True Single Molecule Sequencing (tSMS) technology (e.g. as described in Harris T. D. et al., Science 320:106-109 [2008]). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In certain embodiments the templates can be at a density of about 100 million templates/cm2. The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

In another illustrative, but non-limiting embodiment, the methods described herein comprise obtaining sequence information for the nucleic acids in the test sample, e.g., cfDNA or cellular DNA in a subject being screened for a cancer, using the 454 sequencing (Roche) (e.g., as described in Margulies, M. et al. Nature 437:376-380 [2005]). 454 sequencing typically involves two steps. In the first step, DNA is sheared into fragments of approximately 300-800 base pairs, and the fragments are blunt-ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (e.g., picoliter-sized wells). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is measured and analyzed.

In another illustrative, but non-limiting, embodiment, the methods described herein comprises obtaining sequence information for the nucleic acids in the test sample, e.g., cfDNA or cellular DNA in a subject being screened for a cancer, using the SOLiD™ technology (Applied Biosystems). In SOLiD™ sequencing-by-ligation, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In another illustrative, but non-limiting, embodiment, the methods described herein comprise obtaining sequence information for the nucleic acids in the test sample, e.g., cfDNA or cellular DNA in a subject being screened for a cancer, using the single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector comprises a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In another illustrative, but non-limiting embodiment, the methods described herein comprise obtaining sequence information for the nucleic acids in the test sample, e.g., cfDNA or cellular DNA in a subject being screened for a cancer, using nanopore sequencing (e.g. as described in Soni G V and Meller A. Clin Chem 53: 1996-2001 [2007]). Nanopore sequencing DNA analysis techniques are developed by a number of companies, including, for example, Oxford Nanopore Technologies (Oxford, United Kingdom), Sequenom, NABsys, and the like. Nanopore sequencing is a single-molecule sequencing technology whereby a single molecule of DNA is sequenced directly as it passes through a nanopore. A nanopore is a small hole, typically of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore provides a read of the DNA sequence.

In another illustrative, but non-limiting, embodiment, the methods described herein comprises obtaining sequence information for the nucleic acids in the test sample, e.g., cfDNA or cellular DNA in a subject being screened for a cancer, using the chemical-sensitive field effect transistor (chemFET) array (e.g., as described in U.S. Patent Application Publication No. 2009/0026082). In one example of this technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned as a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In another embodiment, the present method comprises obtaining sequence information for the nucleic acids in the test sample, e.g., cfDNA in a test sample being screened for cancer, using transmission electron microscopy (TEM). The method, termed Individual Molecule Placement Rapid Nano Transfer (IMPRNT), comprises utilizing single atom resolution transmission electron microscope imaging of high-molecular weight (150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA. The method is further described in PCT patent publication WO 2009/046445. The method allows for sequencing complete human genomes in less than ten minutes.

In another embodiment, the DNA sequencing technology is the Ion Torrent single molecule sequencing, which pairs semiconductor technology with a simple sequencing chemistry to directly translate chemically encoded information (A, C, G, T) into digital information (0, 1) on a semiconductor chip. In nature, when a nucleotide is incorporated into a strand of DNA by a polymerase, a hydrogen ion is released as a byproduct. Ion Torrent uses a high-density array of micro-machined wells to perform this biochemical process in a massively parallel way. Each well holds a different DNA molecule. Beneath the wells is an ion-sensitive layer and beneath that an ion sensor. When a nucleotide, for example a C, is added to a DNA template and is then incorporated into a strand of DNA, a hydrogen ion will be released. The charge from that ion will change the pH of the solution, which can be detected by Ion Torrent's ion sensor. The sequencer-essentially the world's smallest solid-state pH meter-calls the base, going directly from chemical information to digital information. The Ion personal Genome Machine (PGM™) sequencer then sequentially floods the chip with one nucleotide after another. If the next nucleotide that floods the chip is not a match. No voltage change will be recorded and no base will be called. If there are two identical bases on the DNA strand, the voltage will be double, and the chip will record two identical bases called. Direct detection allows recordation of nucleotide incorporation in seconds.

In another embodiment, the present method comprises obtaining sequence information for the nucleic acids in the test sample, e.g., cfDNA in a test sample being screened for cancer, using sequencing by hybridization. Sequencing-by-hybridization comprises contacting the plurality of polynucleotide sequences with a plurality of polynucleotide probes, wherein each of the plurality of polynucleotide probes can be optionally tethered to a substrate. The substrate might be flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be determined and used to identify the plurality of polynucleotide sequences within the sample.

In some embodiments of the methods described herein, the mapped sequence tags comprise sequence reads of about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. It is expected that technological advances will enable single-end reads of greater than 500 bp enabling for reads of greater than about 1000 bp when paired end reads are generated. In one embodiment, the mapped sequence tags comprise sequence reads that are 36 bp. Mapping of the sequence tags is achieved by comparing the sequence of the tag with the sequence of the reference to determine the chromosomal origin of the sequenced nucleic acid (e.g., cfDNA) molecule, and specific genetic sequence information is not needed. A small degree of mismatch (0-2 mismatches per sequence tag) may be allowed to account for minor polymorphisms that may exist between the reference genome and the genomes in the mixed sample.

A plurality of sequence tags are typically obtained per sample. In some embodiments, at least about 3×10⁶ sequence tags, at least about 5×10⁶ sequence tags, at least about 8×10⁶ sequence tags, at least about 10×10⁶ sequence tags, at least about 15×10⁶ sequence tags, at least about 20×10⁶ sequence tags, at least about 30×10⁶ sequence tags, at least about 40×10⁶ sequence tags, or at least about 50×10⁶ sequence tags comprising between 20 and 40 bp reads, e.g., 36 bp, are obtained from mapping the reads to the reference genome per sample. In one embodiment, all the sequence reads are mapped to all regions of the reference genome. In one embodiment, the tags that have been mapped to all regions, e.g., all chromosomes, of the reference genome are analyzed, and the SNVs, CNVs or SVs, in the cfDNA sample is determined.

The accuracy required for correctly determining whether a SNV, CNV or SV is present or absent in a sample, is predicated on the variation of the number of sequence tags that map to the reference genome among samples within a sequencing run (inter-chromosomal variability), and the variation of the number of sequence tags that map to the reference genome in different sequencing runs (inter-sequencing variability). For example, the variations can be particularly pronounced for tags that map to GC-rich or GC-poor reference sequences. Other variations can result from using different protocols for the extraction and purification of the nucleic acids, the preparation of the sequencing libraries, and the use of different sequencing platforms. The present method uses sequence doses (chromosome doses, or segment doses) based on the knowledge of normalizing sequences (normalizing chromosome sequences or normalizing segment sequences), to intrinsically account for the accrued variability stemming from interchromosomal (intra-run), and inter-sequencing (inter-run) and platform-dependent variability. Chromosome doses are based on the knowledge of a normalizing chromosome sequence, which can be composed of a single chromosome, or of two or more chromosomes selected from chromosomes 1-22, X, and Y. Alternatively, normalizing chromosome sequences can be composed of a single chromosome segment, or of two or more segments of one chromosome or of two or more chromosomes. Segment doses are based on the knowledge of a normalizing segment sequence, which can be composed of a single segment of any one chromosome, or of two or more segments of any two or more of chromosomes 1-22, X, and Y.

### Apparatus and System for Determining Variant of Interest

Analysis of the sequencing data and the diagnosis derived therefrom are typically performed using various computer executed algorithms and programs. Therefore, certain embodiments employ processes involving data stored in or transferred through one or more computer systems or other processing systems. Embodiments disclosed herein also relate to apparatus for performing these operations. This apparatus may be specially constructed for the required purposes, or it may be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel. A processor or group of processors for performing the methods described herein may be of various types including microcontrollers and microprocessors such as programmable devices (e.g., CPLDs and FPGAs) and non-programmable devices such as gate array ASICs or general purpose microprocessors.

In addition, certain embodiments relate to tangible and/or non-transitory computer readable media or computer program products that include program instructions and/or data (including data structures) for performing various computer-implemented operations. Examples of computer-readable media include, but are not limited to, semiconductor memory devices, magnetic media such as disk drives, magnetic tape, optical media such as CDs, magneto-optical media, and hardware devices that are specially configured to store and perform program instructions, such as read-only memory devices (ROM) and random access memory (RAM). The computer readable media may be directly controlled by an end user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In various embodiments, the data or information employed in the disclosed methods and apparatus is provided in an electronic format. Such data or information may include reads and tags derived from a nucleic acid sample, counts or densities of such tags that align with particular regions of a reference sequence (e.g., that align to a chromosome or chromosome segment), reference sequences (including reference sequences providing solely or primarily polymorphisms), chromosome and segment doses, calls such as SNV or aneuploidy calls, normalized chromosome and segment values, pairs of chromosomes or segments and corresponding normalizing chromosomes or segments, counseling recommendations, diagnoses, and the like. As used herein, data or other information provided in electronic format is available for storage on a machine and transmission between machines. Conventionally, data in electronic format is provided digitally and may be stored as bits and/or bytes in various data structures, lists, databases, etc. The data may be embodied electronically, optically, etc.

One embodiment provides a computer program product for generating an output indicating the presence or absence of an SNV or aneuploidy associated with a cancer, in a test sample. The computer product may contain instructions for performing any one or more of the above-described methods for determining a chromosomal anomaly. As explained, the computer product may include a non-transitory and/or tangible computer readable medium having a computer executable or compilable logic (e.g., instructions) recorded thereon for enabling a processor to determine if a SNV, CNV or SV call should be made. In one example, the computer product comprises a computer readable medium having a computer executable or compilable logic (e.g., instructions) recorded thereon for enabling a processor to diagnose an SNV, CNV or SV.

The sequence information from the sample under consideration may be mapped to chromosome reference sequences to identify a number of sequence tags for each of any one or more chromosomes of interest and to identify a number of sequence tags for a normalizing segment sequence for each of said any one or more chromosomes of interest. In various embodiments, the reference sequences are stored in a database such as a relational or object database, for example.

It should be understood that it is not practical, or even possible in most cases, for an unaided human being to perform the computational operations of the methods disclosed herein. For example, mapping a single 30 bp read from a sample to any one of the human chromosomes might require years of effort without the assistance of a computational apparatus. Of course, the problem is compounded because reliable SNV, CNV or SV calls generally require mapping thousands (e.g., at least about 10,000) or even millions of reads to one or more chromosomes.

The methods disclosed herein can be performed using a system for evaluation of copy number of a genetic sequence of interest in a test sample. The system comprising: (a) a sequencer for receiving nucleic acids from the test sample providing nucleic acid sequence information from the sample; (b) a processor; and (c) one or more computer-readable storage media having stored thereon instructions for execution on said processor to carry out a method for identifying any SNV, CNV or SV.

In some embodiments, the methods are instructed by a computer-readable medium having stored thereon computer-readable instructions for carrying out a method for identifying any SNV, CNV or SV. Thus, one embodiment provides a computer program product comprising one or more computer-readable non-transitory storage media having stored thereon computer-executable instructions that, when executed by one or more processors of a computer system, cause the computer system to implement a method for evaluation of copy number of a sequence of interest in a test sample comprising normal and tumor cell-free nucleic acids. The method includes: (a) retrieving, by the one or more processors, sequence reads and fragment sizes of cfDNA fragments obtained from a test sample; (b) assigning, by the one or more processors, the cfDNA fragments into a plurality of bins representing different fragment sizes; and (c) determining, using the sequence reads and by the one or more processors, an allele frequency of the variant of interest in a prioritized set of bins selected from the plurality of bins, wherein the prioritized set of bins was selected to (i) limit a probability that a quantity of the variant of interest in the prioritized set of bins is below a limit of detection and (ii) increase a probability that a quantity of the variant of interest in the prioritized set of bins is higher than in all bins of the plurality of bins.

In some embodiments, the instructions may further include automatically recording information pertinent to the method such as chromosome doses and the presence or absence of a SNV, CNV or SV in a patient medical record for a human subject providing the biological test sample. The patient medical record may be maintained by, for example, a laboratory, physician's office, a hospital, a health maintenance organization, an insurance company, or a personal medical record website. Further, based on the results of the processor-implemented analysis, the method may further involve prescribing, initiating, and/or altering treatment of a human subject from whom the biological test sample was taken. This may involve performing one or more additional tests or analyses on additional samples taken from the subject.

Disclosed methods can also be performed using a computer processing system which is adapted or configured to perform a method for identifying any SNV, CNV or SV. One embodiment provides a computer processing system which is adapted or configured to perform a method as described herein. In one embodiment, the apparatus comprises a sequencing device adapted or configured for sequencing at least a portion of the nucleic acid molecules in a sample to obtain the type of sequence information described elsewhere herein. The apparatus may also include components for processing the sample. Such components are described elsewhere herein.

Sequence (or other) data can be input into a computer or stored on a computer readable medium either directly or indirectly. In one embodiment, a computer system is directly coupled to a sequencing device that reads and/or analyzes sequences of nucleic acids from samples. Sequences or other information from such tools are provided via interface in the computer system. Alternatively, the sequences processed by system are provided from a sequence storage source such as a database or other repository. Once available to the processing apparatus, a memory device or mass storage device buffers or stores, at least temporarily, sequences of the nucleic acids. In addition, the memory device may store tag counts for various chromosomes or genomes, etc. The memory may also store various routines and/or programs for analyzing the presenting the sequence or mapped data. Such programs/routines may include programs for performing statistical analyses, etc.

In one example, a user provides a sample into a sequencing apparatus. Data is collected and/or analyzed by the sequencing apparatus which is connected to a computer. Software on the computer allows for data collection and/or analysis. Data can be stored, displayed (via a monitor or other similar device), and/or sent to another location. The computer may be connected to the internet which is used to transmit data to a handheld device utilized by a remote user (e.g., a physician, scientist or analyst). It is understood that the data can be stored and/or analyzed prior to transmittal. In some embodiments, raw data is collected and sent to a remote user or apparatus that will analyze and/or store the data. Transmittal can occur via the internet, but can also occur via satellite or other connection. Alternately, data can be stored on a computer-readable medium and the medium can be shipped to an end user (e.g., via mail). The remote user can be in the same or a different geographical location including, but not limited to a building, city, state, country or continent.

In some embodiments, the methods also include collecting data regarding a plurality of polynucleotide sequences (e.g., reads, tags and/or reference chromosome sequences) and sending the data to a computer or other computational system. For example, the computer can be connected to laboratory equipment, e.g., a sample collection apparatus, a nucleotide amplification apparatus, a nucleotide sequencing apparatus, or a hybridization apparatus. The computer can then collect applicable data gathered by the laboratory device. The data can be stored on a computer at any step, e.g., while collected in real time, prior to the sending, during or in conjunction with the sending, or following the sending. The data can be stored on a computer-readable medium that can be extracted from the computer. The data collected or stored can be transmitted from the computer to a remote location, e.g., via a local network or a wide area network such as the internet. At the remote location various operations can be performed on the transmitted data as described below.

Among the types of electronically formatted data that may be stored, transmitted, analyzed, and/or manipulated in systems, apparatus, and methods disclosed herein are the following: Reads obtained by sequencing nucleic acids in a test sample Tags obtained by aligning reads to a reference genome or other reference sequence or sequences The reference genome or sequence Sequence tag density—Counts or numbers of tags for each of two or more regions (typically chromosomes or chromosome segments) of a reference genome or other reference sequences Identities of normalizing chromosomes or chromosome segments for particular chromosomes or chromosome segments of interest Doses for chromosomes or chromosome segments (or other regions) obtained from chromosomes or segments of interest and corresponding normalizing chromosomes or segments Thresholds for calling chromosome doses as either affected, non-affected, or no call The actual calls of chromosome doses Diagnoses (clinical condition associated with the calls) Recommendations for further tests derived from the calls and/or diagnoses Treatment and/or monitoring plans derived from the calls and/or diagnoses

These various types of data may be obtained, stored transmitted, analyzed, and/or manipulated at one or more locations using distinct apparatus. The processing options span a wide spectrum. At one end of the spectrum, all or much of this information is stored and used at the location where the test sample is processed, e.g., a doctor's office or other clinical setting. In other extreme, the sample is obtained at one location, it is processed and optionally sequenced at a different location, reads are aligned and calls are made at one or more different locations, and diagnoses, recommendations, and/or plans are prepared at still another location (which may be a location where the sample was obtained).

In various embodiments, the reads are generated with the sequencing apparatus and then transmitted to a remote site where they are processed to produce calls. At this remote location, as an example, the reads are aligned to a reference sequence to produce tags, which are counted and assigned to chromosomes or segments of interest. Also at the remote location, the counts are converted to doses using associated normalizing chromosomes or segments. Still further, at the remote location, the doses are used to generate calls.

Among the processing operations that may be employed at distinct locations are the following: Sample collection Sample processing preliminary to sequencing Sequencing Analyzing sequence data and deriving SNV, CNV or SV calls Diagnosis Reporting a diagnosis and/or a call to patient or health care provider Developing a plan for further treatment, testing, and/or monitoring Executing the plan Counseling.

Any one or more of these operations may be automated as described elsewhere herein. Typically, the sequencing and the analyzing of sequence data and deriving SNV, CNV or SV calls will be performed computationally. The other operations may be performed manually or automatically.

Examples of locations where sample collection may be performed include health practitioners' offices, clinics, patients' homes (where a sample collection tool or kit is provided), and mobile health care vehicles. Examples of locations where sample processing prior to sequencing may be performed include health practitioners' offices, clinics, patients' homes (where a sample processing apparatus or kit is provided), mobile health care vehicles, and facilities of SNV, CNV or SV analysis providers. Examples of locations where sequencing may be performed include health practitioners' offices, clinics, health practitioners' offices, clinics, patients' homes (where a sample sequencing apparatus and/or kit is provided), mobile health care vehicles, and facilities of SNV, CNV or SV analysis providers. The location where the sequencing takes place may be provided with a dedicated network connection for transmitting sequence data (typically reads) in an electronic format. Such connection may be wired or wireless and have and may be configured to send the data to a site where the data can be processed and/or aggregated prior to transmission to a processing site. Data aggregators can be maintained by health organizations such as Health Maintenance Organizations (HMOs).

The analyzing and/or deriving operations may be performed at any of the foregoing locations or alternatively at a further remote site dedicated to computation and/or the service of analyzing nucleic acid sequence data. Such locations include for example, clusters such as general purpose server farms, the facilities of an SNV, CNV or SV analysis service business, and the like. In some embodiments, the computational apparatus employed to perform the analysis is leased or rented. The computational resources may be part of an internet accessible collection of processors such as processing resources colloquially known as the cloud. In some cases, the computations are performed by a parallel or massively parallel group of processors that are affiliated or unaffiliated with one another. The processing may be accomplished using distributed processing such as cluster computing, grid computing, and the like. In such embodiments, a cluster or grid of computational resources collective form a super virtual computer composed of multiple processors or computers acting together to perform the analysis and/or derivation described herein. These technologies as well as more conventional supercomputers may be employed to process sequence data as described herein. Each is a form of parallel computing that relies on processors or computers. In the case of grid computing these processors (often whole computers) are connected by a network (private, public, or the Internet) by a conventional network protocol such as Ethernet. By contrast, a supercomputer has many processors connected by a local high-speed computer bus.

In certain embodiments, the diagnosis is generated at the same location as the analyzing operation. In other embodiments, it is performed at a different location. In some examples, reporting the diagnosis is performed at the location where the sample was taken, although this need not be the case. Examples of locations where the diagnosis can be generated or reported and/or where developing a plan is performed include health practitioners' offices, clinics, internet sites accessible by computers, and handheld devices such as cell phones, tablets, smart phones, etc. having a wired or wireless connection to a network. Examples of locations where counseling is performed include health practitioners' offices, clinics, internet sites accessible by computers, handheld devices, etc.

In some embodiments, the sample collection, sample processing, and sequencing operations are performed at a first location and the analyzing and deriving operation is performed at a second location. However, in some cases, the sample collection is collected at one location (e.g., a health practitioner's office or clinic) and the sample processing and sequencing is performed at a different location that is optionally the same location where the analyzing and deriving take place.

In various embodiments, a sequence of the above-listed operations may be triggered by a user or entity initiating sample collection, sample processing and/or sequencing. After one or more these operations have begun execution, the other operations may naturally follow. For example, the sequencing operation may cause reads to be automatically collected and sent to a processing apparatus which then conducts, often automatically and possibly without further user intervention, the sequence analysis and derivation of SNV, CNV or SV operation. In some implementations, the result of this processing operation is then automatically delivered, possibly with reformatting as a diagnosis, to a system component or entity that processes and reports the information to a health professional and/or patient. As explained such information can also be automatically processed to produce a treatment, testing, and/or monitoring plan, possibly along with counseling information. Thus, initiating an early-stage operation can trigger an end to end sequence in which the health professional, patient or other concerned party is provided with a diagnosis, a plan, counseling and/or other information useful for acting on a physical condition. This is accomplished even though parts of the overall system are physically separated and possibly remote from the location of, e.g., the sample and sequence apparatus.

### Overview

Preoperative diagnosis of ovarian cancer remains challenging. More accurate detection of ovarian cancer at an earlier stage would reduce the number of unnecessary surgeries on benign pelvic masses. Currently, tumor markers and imaging characteristics offer only limited sensitivity and specificity, and surgical excision is the only definitive way to achieve diagnosis.

In certain aspects of the present invention, a system has been developed that used single nucleotide variations (SNVs), copy number variations (CNVs) or structural variations (SVs) in patient DNA to distinguish ovarian cancer from benign fallopian tubes (the location from which ovarian cancer arises). The system successfully makes this distinction with 100% accuracy. It has been validated both internally and externally with retention of its accuracy.

Briefly, a biological sample is taken from a patient, and target SNVs, CNVs and/or SVs present in circulating tumor DNA (ctDNA) found in the sample are identified to generate a “patient signature.” The patient signature is compared to a “diagnostic panel” of particular SNVs, CNVs and/or SVs known to correlate with ovarian cancer. If eighteen or more diagnostic model SNVs, CNVs and/or SVs are present in the patient signature, a high-certainty diagnosis is made that the patient has ovarian cancer or has a recurrence of ovarian cancer. Thus, a high-certainty diagnosis is made with a simple, peripheral blood draw. Based on the diagnosis, appropriate treatment is commenced. This method is useful for diagnosis in patients with a pelvic mass, for screening high-risk populations, or in surveillance for ovarian cancer recurrence.

### Biological Samples

Since blood contacts most of tumors, liquid biopsies (LBs) mostly involve blood sampling, although other body fluids like mucosa, pleural effusions, urine, and cerebrospinal fluid (CSF) are also analyzed. In certain embodiments, a biological sample is obtained from a patient. In certain embodiments, the sample is a liquid biopsy, such as a blood or plasma sample. In certain embodiments, the sample is mucosa, pleural effusions, urine, and cerebrospinal fluid (CSF).

In certain embodiments, the sample contains circulating tumor cells (CTCs) that are shed by both primary and metastatic tumors, circulating tumor DNA (ctDNA), tumor derived extracellular vesicles (EVs) that are membrane-bound subcellular moieties composed of nucleic acids/proteins, tumor educated platelets (TEPs), and circulating cell-free RNA (cfRNA), composed of small RNAs/miRNAs.

### Circulating Tumor Cells in Liquid Biopsies

Circulating tumor cells (CTCs) are initially released from primary tumors in the tissue, travel through the circulatory system and account for the development of metastatic (or secondary) tumors at distant sites in the body. The percentage in the blood is quite low, with nearly one CTC found per million leukocytes. Various technologies have been used to selectively detect viable CTCs to obtain information regarding tumors. One example is the EPISPOT (EPithelialImmunoSPOT) assay that detects circulating tumor cells up to a single cell. The assay involves the use of membrane-bound antibodies against the epithelial cell adhesion molecule (EpCAM, or CD326) present on tumor cells and their subsequent culturing/expansion in both in vivo and in vivo conditions. Another positive selection/enrichment technology for CTCs obtained from LB samples is the CellSearch system. This technology uses antibody-labeled magnetic beads to pull down CTCs with epithelial lineage markers (like EpCAM). Another immunomagnetic-based enrichment assay of CTCs from LBs is the AdnaTest. In addition to the EpCAM-labeled ferromagnetic beads used in the CellSearch system, AdnaTest includes a polymerase chain reaction (PCR) step to detect tumor-specific mRNA transcripts.

Alternative approaches include microfluidic devices used to select CTCs in various types of cancers. Devices like the “CTC-Chip,” which contains thousands of small antibody-labeled microposts, have been used to capture CTCs bearing specific tumor antigens from LB blood samples. Certain designs of “CTC-Chips” have been demonstrated to employ patterns of microgrooves, which seem to increase the contact time between antibody-labeled microposts and CTCs, improving cellular entrapment. CTCs filtered off from LB samples by the chip are then imaged and analyzed.

Functional assays like the Metastasis-Initiating-Cells (MIC) assay analyze the invasive properties of CTCs obtained from LB into the surrounding matrix in vivo, assisting in their further characterization. These analyses aid in providing a detailed picture of tumor staging/subtypes and in designing novel personalized therapeutic drugs against tumors. In addition to general nuclear and surface-specific markers targeting CTCs, counterstain markers that target cells in exclusion to CTCs such as white blood cells (WBCs), platelets, red blood cells (RBCs), etc., can also be used to enrich CTCs from blood samples. The prominent markers selected for counterstains include CD45/CD66b (granulocytes), CD235a (RBCs), CD41/CD61 (platelets), CD4/CD8 (lymphocytes), CD11b/CD14 (macrophages) and CD34 (hematopoietic progenitors/endothelial cells). Technologies like the EasySep Depletion Kit (StemCell Technologies) use CD45-labeled magnetic beads to negatively select WBCs, depleting them from the LB samples. Other examples, like the RosetteSep (StemCell Technologies) method, use an additional density gradient centrifugation step for further CTC enrichment.

Post-enrichment technologies, such as the DEPArray™ System, have also been demonstrated to successfully isolate and recover single CTCs from LB samples of whole blood. Next-generation sequencing (NGS) analysis can be carried out directly on CTCs using technologies such as the Ion Torrent PGM™ system, composed of the Ion AmpliSeq™ Cancer Hotspot Panel, which provides enhanced mutational analysis and avoids the use of error-prone methods like whole genome amplification (WGA), thus improving screening accuracy.

In certain embodiments, circulating tumor cells are present in the liquid biopsy; i.e., whole tumor cells are present, and not just DNA fragments.

### Circulating Tumor DNA in Liquid Biopsies

In certain embodiments, circulating tumor DNA (ctDNA) is present in the liquid biopsy. Over time, fragments of DNA from the tumor cells can enter a patient's bloodstream, and this DNA is called circulating tumor DNA (ctDNA). This ctDNA can be from dying tumor cells or as the cancer cells turnover. Circulating tumor DNA (ctDNA) is single- or double-stranded DNA released by the tumor cells into the blood and it thus harbors the mutations of the original tumor. Circulating tumor DNA (ctDNA) is distinguishable from cell-free DNA (cfDNA), in that DNA fragments shed from non-tumor cells are cfDNA, whereas DNA fragments shed from tumors are ctDNA. ctDNA accounts for about 0.1-10% of the total circulating cell-free DNA (cfDNA). ctDNA levels in plasma, however, can vary depending on tumor load, tumor stage, and therapeutic response. Recent studies have shown that ctDNA differs in length from the cfDNA, with reports indicating ctDNA fractions in patients with cancer to be 20-50 base pairs, which is generally shorter than cfDNA.

Two major types of approaches have been considered for ctDNA analysis: targeted approaches that focus on specific gene rearrangements or gene mutations in particular genomic regions that act as “hotspots” for variation in a given tumor type, or untargeted approaches that offer a broader analysis and monitoring of the tumor genome, providing information on nucleotide alterations, copy number aberrations, chromosomal alterations, etc., independent of any prior data on molecular alterations.

Targeted approaches include PCR-based methods such as droplet digital PCR and BEAMing that have shown remarkable sensitivity of 1 to 0.001% in detecting somatic point mutations. Droplet digital PCR involves partitioning the sample DNA (target and background DNA) into numerous independent partitions or droplets. The target sequence is then amplified by end point PCR in each droplet and relative fractions of positive and negative droplets counted (fluorescent probes) that provide relative quantification of target samples.

BEAMing (beads, emulsions, amplification, and magnetics), on the other hand, is a modification of emulsion PCR where several different templates are amplified within a single tube, each in different compartments (or emulsion droplets) but along with primer bound beads that are recovered with the help of a magnetic field or centrifugal force. PCR-based assays that detect genomic rearrangements explicitly associated with the tumor genome have shown promising results in sensitivity and specificity using ctDNA. Assays such as personalized analysis of rearranged ends (PARE), which uses primers flanking the breakpoint region, have been shown to successfully detect mutant ctDNA (rearranged sequences) at levels as low as 0.001% in plasma samples of patients. PARE analysis of ctDNA assists in monitoring disease burden and the development of tumor-specific biomarkers in patients with solid tumors. Numerous NGS-based methods have recently been developed that offer a relatively broader screening of the genomic regions, along with better resolutions in detecting mutations in ctDNA samples. Assays such as tagged-amplicon deep sequencing (TAm-Seq) can detect ctDNA mutations in plasma with very low allelic frequencies (~2%) and with high sensitivity (>97%). Various sequence-specific primers first amplify multiple regions of the targeted area in the genome to allow the representation of various alleles in the template material, narrowing down the pool of amplified products. These diverse products are again amplified for enrichment, tagged with adaptors, and sequenced. Further, TAm-Seq has also assisted in the longitudinal screening of tumor mutations over several months in plasma of patients. Similar deep sequencing methods like CAPP-Seq have been developed that allowed the detection of ctDNA mutant fractions as low as 0.02% with high specificity (~95%) in patients. ctDNA quantified by CAPP-Seq analysis has been shown to be better in correlating to tumor burden, detecting residual disease and accessing an early tumor response than traditional radiographic methods. Tagged complementary oligonucleotide probes that can be recovered are used to target specific regions of DNA.

In contrast to the targeted approaches discussed above that focus on primer-specified regions, untargeted methods are relatively more comprehensive about analyzing the tumor genome. In this context, methods such as shotgun massively parallel sequencing of ctDNA from plasma have been shown to provide whole-genome profiling for copy number alterations (CNA) and mutations in patients. Similar whole-genome profiling of plasma ctDNA using high-throughput IlluminasMiSeq has been used. Whole-genome analysis using massively parallel sequencing of plasma ctDNA has also enabled the detection of similar alterations in patients.

### Non-Blood Liquid

In addition to circulatory fluids like plasma or serum, other body fluids such as saliva and urine can be used as liquid biopsies. Saliva offers practical advantages with regard to ease of access, non-invasiveness, and cost effectiveness in sampling, even more so than plasma or serum. Novel electrochemical sensor-based technologies like an electric field-induced release and measurement (EFIRM) have been shown to detect EGFR mutations (tyrosine kinase domain) from bodily fluids like saliva in patients. Similar EFIRM based technologies have been used in developing salivary biomarkers.

The completely non-invasive nature of urine sampling, relative to tissue or even blood, makes it a quite useful candidate in LBs, particularly in cases where repeated sampling is required to monitor tumor progression and therapeutic outcomes.

### Probes and Diagnostic Panels

In certain embodiments, the present invention provides a diagnostic panel of probes that hybridize to nucleic acid. In certain embodiments the nucleic acid is ctDNA. In certain embodiments, the panel comprises at least 18 probes specific for single nucleotide variations (SNV) selected from the group consisting of the following:

| | | | | SNV |
|---|---|---|---|---|
| Loci | Allele | Gene | RS | CLASS |
| chr1_KI270706v1_random: 30898 | G | — | — | SNV |
| chr1_KI270706v1_random: 4207 | T | — | — | SNV |
| chr1_KI270707v1_random: 16224-16225 | — | — | — | deletion |
| chr1_KI270709v1_random: 8467 | A | — | — | SNV |
| chr1_KI270711v1_random: 37219-37220 | A | — | — | insertion |
| chr1_KI270712v1_random: 56231 | G | — | — | SNV |
| chr1_KI270712v1_random: 58298 | A | — | — | SNV |
| chr1_KI270713v1_random: 28405 | A | ENSG00000277475 | — | SNV |
| chr1_KI270713v1_random: 29554-29555 | T | ENSG00000277475 | — | insertion |
| chr1_KI270713v1_random: 30395 | G | ENSG00000277475 | — | SNV |
| chr1_KI270714v1_random: 33537-33540 | — | — | — | deletion |
| chr1: 100138524 | C | ENSG00000122435 | rs834163 | SNV |
| chr1: 100146015 | T | ENSG00000122477 | rs12733952 | SNV |
| chr1: 100146375 | G | ENSG00000122477 | rs506044 | SNV |
| chr1: 100151816 | A | ENSG00000122477 | rs571579 | SNV |
| chr1: 100266264 | A | ENSG00000137996 | rs57327535 | SNV |
| chr1: 100400148 | G | ENSG00000079335 | rs654970 | SNV |
| chr1: 100446649 | — | ENSG00000079335 | rs57522935 | deletion |
| chr1: 100991465 | T | ENSG00000117543 | rs1188734 | SNV |
| chr1: 105560508 | G | — | rs1721558 | SNV |
| chr1: 112623613 | A | ENSG00000116489 | rs11102517 | SNV |
| chr1: 112707780 | C | ENSG00000155366 | rs3125045 | SNV |
| chr1: 11985866 | G | ENSG00000116688 | rs2336383 | SNV |
| chr1: 121116927 | C | ENSG00000213244 | rs182963377 | SNV |
| chr1: 121570138-121570139 | T | ENSG00000224857 | rs35074347 | insertion |
| chr1: 1306420 | G | ENSG00000131584 | rs3737720 | SNV |
| chr1: 144426370 | C | ENSG00000266338 | rs148988580 | SNV |
| chr1: 145405191 | G | ENSG00000162825 | rs373112567 | SNV |
| chr1: 145405199 | T | ENSG00000162825 | rs376281416 | SNV |
| chr1: 147214459-147214460 | A | ENSG00000131781 | rs782281901 | insertion |
| chr1: 147244486-147244487 | T | ENSG00000131778 | rs1181393925 | insertion |
| chr1: 154582475-154582476 | GGGGCATG | ENSG00000160710 | rs113414804 | insertion |
| chr1: 154598698-154598699 | GGA | ENSG00000160710 | rs4045821 | insertion |
| chr1: 43746463 | — | ENSG00000126091 | rs35545005 | deletion |
| chr10: 75219117 | C | ENSG00000165637 | — | SNV |
| chr10: 77169216 | A | ENSG00000156113 | rs481869 | SNV |
| chr10: 77169959 | A | ENSG00000156113 | rs1265491 | SNV |
| chr10: 77347376 | T | ENSG00000156113 | rs207676 | SNV |
| chr10: 87060764 | A | ENSG00000148672 | — | SNV |
| chr10: 87311353-87311356 | — | ENSG00000223482 | rs79207971 | deletion |
| chr10: 94108363 | T | ENSG00000138193 | rs1925246 | SNV |
| chr11: 46376342 | A | ENSG00000149091 | rs200365833 | SNV |
| chr11: 47258459 | G | ENSG00000025434 | rs326220 | SNV |
| chr11: 60183592 | G | ENSG00000110077 | rs581818 | SNV |
| chr12: 54281845 | G | ENSG00000094916 | — | SNV |
| chr12: 54282228 | A | ENSG00000094916 | rs985359230 | SNV |
| chr12: 6869295 | C | ENSG00000111667 | — | SNV |
| chr21: 33576384 | C | ENSG00000159147 | rs200247542 | SNV |
| chr21: 37159760 | C | ENSG00000182670 | — | SNV |

In certain embodiments, the panel comprises at least 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 SNV probes. In certain embodiments, the panel comprises 49 SNV probes.

In certain embodiments, the panel comprises at least 2 probes specific for copy number variations (CNV) selected from the group consisting of the following:

| Transcript | chr | start | end |
|---|---|---|---|
| ENST00000438676 | 6 | 75290253 | 75290338 |
| ENST00000607397 | 8 | 67345126 | 67345181 |
| ENST00000642833 | 8 | 10474564 | 10474631 |
| MIR2113 | 6 | 98024530 | 98024619 |
| MIR30C2 | 6 | 71376959 | 71377031 |
| MIR3144 | 6 | 120015178 | 120015257 |
| MIR3156-3 | 21 | 13406383 | 13406460 |
| MIR384 | X | 76919272 | 76919360 |
| MIR4643 | 6 | 91521659 | 91521737 |
| MIR548AG1 | 4 | 60922618 | 60922684 |
| MIR8056 | 5 | 173347454 | 173347536 |

In certain embodiments, the panel comprises at least 2, 3, 4, 5, 6, 7, 8, 9, 10, or 11 CNV probes. In certain embodiments, the panel comprises 11 CNV probes.

In certain embodiments, the panel comprises at least 17 probes specific for structural variations (SV) selected from the group consisting of the following:

SV

chr10_101729717_NE
chr11_2149345_PNJ
chr11_89498187_AS
chr13_55179004_NE
chr14_105743009_NE
chr14_105863198_NE
chr14_105863814_UN
chr14_105986593_FUS
chr19_8876368_IGR
chr20_33053965_NEJ
chr21_8259383_NE
chr3_147402104_RI
chr5_102458491_AS
chr5_102608496_INS
chr5_148089622_EE
chr5_85663902_NEJ
chr6_170156045_AS

In certain embodiments, the panel comprises 17 or more SV probes.

In certain embodiments, each probe in the panel of probes is operably linked to a genotyping microchip.

### Methods of Detecting Biomarkers Specific for Ovarian Cancer

First a blood sample is taken and cfDNA is extract from it. Then, genomic regions are amplified around the 49 SNVs via standard PCR on a thermal cycler using primers specific for those regions. Next, the products are hybridize to a custom chip with reference and alternative alleles of each of these loci. The resulting genotypes are analyzed for risk of ovarian cancer. If all alternative (49) SNV are present, then the patient has a 100% risk of presenting ovarian cancer, if none of the alternative SNVs are present, then the risk is 0%. The risk of ovarian cancer is assessed by the model developed in this study and the number and type of alternative SNVs present.

### Methods of Treating a Patient Identified as Having Biomarkers Specific for Ovarian Cancer

Once the patient signature is determined for the ctDNA found in the sample and compared to the ovarian cancer “diagnostic panel” appropriate treatment is commenced. [If the final risk of Ovarian cancer is over 33% by the “diagnostic panel” further study is recommend either by radiology or surgery depending the surgical risk of the patient. If the surgical risk is minimal and the ovarian risk is above the cut-off (33%), a conversation would be initiated with the patient about potential removal of tubes and ovaries for ovarian cancer risk reduction.

The invention will now be illustrated by the following non-limiting Examples.

### Example 1

### Using Genomic Variation to Diagnose Ovarian Cancer: The Path to Liquid Biopsy

### Introduction

Ovarian cancer remains one of the most deadly cancers in women in the United States because it is diagnosed in an advanced stage in over 70% of patients, with 5-year overall survival around 40-50%. Patients diagnosed at early stages have significantly improved 5-year survival exceeding 90%. High grade serous carcinoma (HGSC) is the most common histologic type of the ovarian cancer spectrum, that includes fallopian tube, ovarian and primary peritoneal cancers. It is clear that early diagnosis of tubo-ovarian cancer would save lives. Unfortunately, no current method of screening has been proven effective at detecting ovarian cancer at an earlier stage.

Patients with ovarian cancer typically present with a pelvic mass, with or without symptoms or evidence of metastasis. In those without evidence of metastasis, it is difficult to determine if the mass is benign or malignant preoperatively. A biopsy of the mass is not advised as it has the potential to upstage the patient and may or may not obtain adequate tissue for diagnosis. Numerous medical organizations and groups have published recommendations on how to manage these pelvic masses. Usually, clinical data, tumor markers and imaging studies (generally ultrasound) are used. Regrettably, the specificity of these algorithms varies depending on the utilized methods, and a large proportion of these masses are removed surgically, even when found by chance in asymptomatic women. Based on the available evidence, the US Preventive Services Task Force (USPSTF) concluded that given the lack of mortality benefit of screening, and the moderate to substantial harms that could result from false-positive screening test results and subsequent surgery screening for ovarian cancer in asymptomatic women is not recommended.

The current standard to treat any suspicious masses is to surgically remove them, leading to surgery on a large number of benign masses, which could have been safely observed, an expensive strategy to reduce ovarian cancer mortality. It is estimated that for every 10 women that undergo surgery for a pelvic mass, only one would be diagnosed with ovarian cancer. Furthermore, over 10% of these patients undergoing surgery will experience a major complication. It has been repeatedly shown that ovarian cancer patients treated by trained gynecologic oncologists have improved outcomes over patients treated by other practitioners. These specialists are thoroughly trained in the surgical and medical management of this disease, but they are a limited resource. It is estimated that a third of patients with gynecological malignancies never see a gynecologic oncologist. As a result, it is critical to better select patients with a high risk of ovarian cancer to be referred to trained specialists.

Cancers undergo myriad mutations and alterations in their genome during their development. These changes can be identified in circulating tumor DNA (ctDNA) or circulating tumor cells (CTC) in patient plasma, which is the basis for “liquid biopsies.” This method has been used to identify therapeutically significant mutations in lung cancer to drive treatment strategy, as well as for early diagnosis in various cancers. A liquid biopsy has the potential to diagnose ovarian cancer in early stages and improve overall survival. A large biobank of ovarian cancer specimens and normal fallopian tubes were leveraged to analyze various genomic variations in HGSC. Genetic variation of HGSC and normal fallopian tubes were assessed and prediction models were created to accurately distinguish HGSC from benign tissue. Then, those models were validated with different platforms, samples, and some of them with machine learning algorithms.

### Methods

A single institution, retrospective cohort study was performed using HGSC tumor specimens obtained at the time of cytoreductive surgery and compared them to benign fallopian tube specimens collected at the time of surgery for benign indications. DNA and RNA were isolated from all specimens and whole exome sequencing (WES) and RNA sequencing (RNA-seq) were performed. Using this WES and RNA-seq data, single nucleotide variants (SNV), copy number variation (CNV) and structural variation (SV) were identified.

Genetic variation was identified initially from WES performed in DNA extracted from both HGSC (n=20) and normal Fallopian tubes (N=14). Differences between variants from HGSC and tubes were assessed. Then, models to predict HGSC were constructed using multivariate lasso regression analysis with variants that were different between HGSC and tubes. These models were then validated with RNA-seq data from 112 HGSC cases and 12 controls (tubes), and with RNA-seq from The Cancer Genome Atlas (TCGA) database (all HGSC, N=376). Prediction models were further validated using machine learning algorithms with TensorFlow.

### Specimen Acquisition

HGSC tissue samples and clinical outcome data were obtained from the Department of Obstetrics and Gynecology Gynecologic Oncology Biobank (IRB, ID #200209010), which is part of the Women's Health Tissue Repository (WHTR, IRB, ID #201804817). All specimens archived in the Gynecologic Oncology Biobank (herein termed Biobank) were originally obtained from adult patients under informed consent in accordance with University of Iowa (UI) IRB guidelines. Tumor samples were collected, reviewed by a board-certified pathologist, flash-frozen, and then the diagnosis was confirmed in paraffin at the time of initial surgery. All experimental protocols were approved by the University of Iowa (UI) Biomedical IRB-01.

Fallopian tube samples were then collected from women undergoing gynecologic procedures. Fallopian tubes were obtained from patients with no family history of cancer beside squamous cell carcinoma of the skin and who were undergoing salpingectomy for benign indications (mainly sterilization). Fallopian tubes were chosen as controls as this is the most likely origin of HGSC. DNA and RNA were extracted from epithelial tissue coming from the junction of the ampullary and fimbriated end of fallopian tubes. Twenty normal fallopian tube specimens were obtained. Of those, 12 produced viable RNA for analysis. RNA from both the fallopian tube and HGSC specimens had already been extracted and purified in a previous study. WES was performed on 14 fallopian tubes.

483 patients with serous ovarian cancer were identified in the database. Only patients with a primary diagnosis of advanced Stage (III and IV) HGSC were included. Of these, 112 patients with fresh frozen specimens were selected for RNA isolation and genomic analysis, as described previously. 20 patients with specimens with high quality DNA were also selected for WES to perform variation analysis.

### DNA Sequencing

Genomic DNAs (gDNAs) were purified from frozen tumor and fallopian tube tissues using the DNeasy Blood and Tissue Kit according to manufacturer's (QIAGEN) recommendations. Yield and purity were assessed on a NanoDrop Model 2000 spectrophotometer and by horizontal agarose gel electrophoresis. Whole exome sequencing (WES) was performed externally by GeneWiz (Azenta, Chelmsford, MA) with 100× coverage. Mean quality score was 37.76 and the percent of bases greater than or equal to 30 was 89.96.

### RNA Sequencing

RNA was isolated from the tumor and control specimens in the Biobank. RNA extraction, processing and sequencing have been described previously. In brief, total cellular RNA was extracted from primary tumor tissue using the mirVana (Thermo Fisher, Waltham, USA) RNA purification kit. The RNA yield and quality were assessed with Trinean Dropsense 16 spectrophotometer and Agilent Model 2100 bioanalyzer. RNA quality was determined to be adequate if the sample had an RNA integrity number (RIN) of 7.0 or greater. Samples that were of adequate quality were then sequenced. 500 ng of RNA was quantified by Qubit measurement (Thermo Fisher). RNA was then converted to cDNA and ligated to sequencing adaptors with Illumina TriSeq stranded total RNA library preparation (Illumina, San Diego, CA, USA). cDNA samples were then sequenced with the Illumina HiSeq 4000 genome sequencing platform using 150 bp paired-end SBS chemistry. All sequencing was performed at the Genome Facility at the University of Iowa Institute of Human Genetics (IIHG).

### Single Nucleotide Variation (SNV) Analysis

DNA from WES was aligned to the human reference genome (version hg38) using the SubRead suite. BAM files resulting from the alignment were used with samtools and VarScan software to create Variant Call Format (VCF) files for further analysis. Two separate methods were used to identify all possible SNV's present in VCF files. The first method used was Ensembl Variant Effect Predictor (VEP). This method determines if variants cause significant downstream changes in the genome. Variants present upstream of transcripts, coding regions, regulatory regions, non-coding RNA, and that have downstream consequences (i.e., missense, frameshift, stop gained or lost) are retained and inconsequential variants are removed. A table with present SNVs for all samples was constructed and used for analyses.

In parallel, VCF files were processed with superFreq package to detect other novel SNVs. This package uses a series of filters to discard SNV with lesser quality. Also, SNV detected by superFreq that were present in the gnomAD database were removed. SuperFreq assessed SNV in each transcript of each sample and performed a log-ratio of transcripts with versus without SNV within that sample. That log Ratio was used later for further comparative analyses. All unique loci containing those SNV's by both methods were codified.

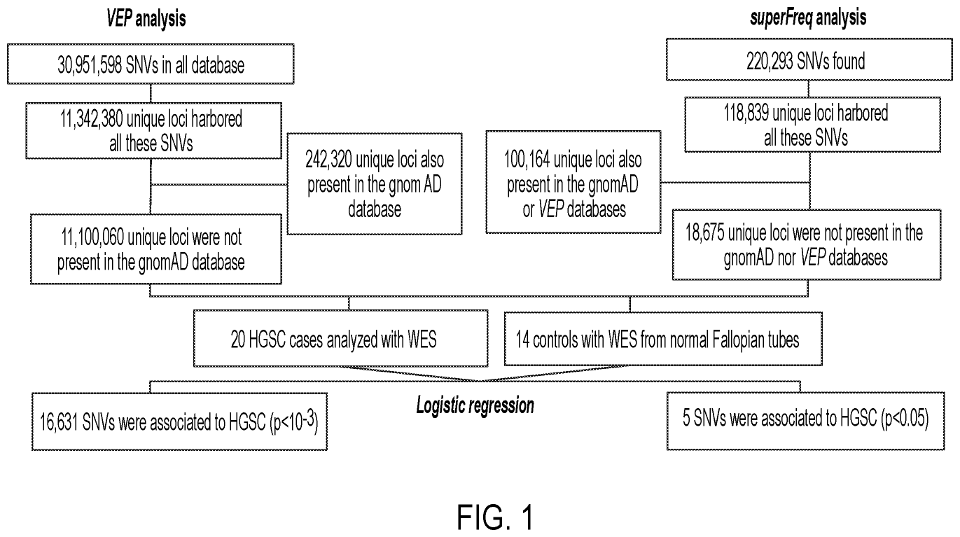

To identify those SNV unique in HGSC, all common SNV present in a large, publicly available database, gnomAD, were subtracted from SNV results from both VEP and superFreq (FIG. 1). The gnomAD database (gnomad.broadinstitute.org) is a database containing 125,748 WES from various studies, aggregated by researchers worldwide. Multiple univariate logistic regression analyses with each of the resulting SNVs were performed to identify those variants more informative for cancer (HGSC, a dichotomous dependent variable, y). The goal was to reduce the number of variables to be introduced in the multivariate prediction analysis to build a classifier. Independent variables (x) were the presence (or absence) of a particular SNV. The cut-off level was established at a p-value <0.001. Using the variants identified in the univariate analysis, an enrichment pathway analysis was performed using the clusterProfiler R package, which interrogates the KEGG database (https://www.genome.jp/kegg/pathway.html). Significance at the p-value level of <0.05 were corrected for multiple comparisons using false discovery rate (FDR). Variants selected with these univariate analyses were included in a multivariate lasso regression analysis to create prediction models of HGSC. Performance of all prediction models were measured by the area under the curve (AUC).

Validation was performed in two different datasets: with RNA-seq data from UI specimens and with RNA-seq data downloaded from The Cancer Genome Atlas (TCGA). UI RNA-seq data was also aligned to the human reference genome (version hg38) with the STAR suite. As before, BAM files were used to create VCF files with samtools and VarScan, and SNVs were detected with both VEP and superFreq. SNVs from RNA-seq data were compared to those identified in DNA through WES. Only SNVs from the RNA-seq experiments that were present and significant in the WES univariate logistic analysis of HGSC versus normal tubes were used for validation analysis. These common SNVs were assessed for their association with ovarian cancer (HGSC) in multiple univariate logistic models (cut-off, p-value <0.05). As before, selected SNVs in univariate analyses were used to create prediction models of HGSC using lasso regression. In addition, pathway enrichment analysis using the KEGG database was performed with these significant SNVs in the univariate analysis.

After permission was granted to access controlled data by the Genomic Data Commons (GDC) Data Portal (dbGaP #29868), TCGA HGSC BAM files from RNA-seq experiments aligned to the human reference genome (version hg38) with the STAR suite were downloaded in their original format. TCGA contains RNA-seq data but lacks RNA-seq from normal tubal samples. It was possible, though, to create VCF files from original TCGA BAM files. SNVs were identified by performing VEP and superFreq analyses, in the same way as the WES analysis. Then, these SNVs were compared to those present and significantly associated with HGSC in the WES analysis.

To further validate the analysis, the prediction analysis was performed in a machine learning platform. For that purpose, TensorFlow waw used in a Jupyter notebook with a Keras application programming interface (API) (28). TensorFlow code was modified from a tutorial. Several models were tested in this platform: first, the model with all SNV associated with HGSC, the model resulting from the multivariate analysis with lasso, the model for the validation with IU RNA-seq SNVs, and the model for the validation with RNA-seq extracted from TCGA. We used classification machine learning approaches to discriminate tube versus HGSC. Training and testing were performed to account for weights of the outcomes as well as for unbalanced data (fewer controls than cases for both DNA and RNA samples).

### Copy Number Variant (CNV) Analysis

Resulting DNA WES files from the Subread alignment (BAM) were assessed for copy number variations using superFreq. Once CNV were detected, a univariate logistic regression was carried out to identify CNV differences between HGSC and control samples, with a p-value of <0.001 to account for multiple comparisons. Enrichment pathway analysis was performed with genes that had significant CNV differences in the univariate analysis using clusterProfiler and the KEGG database. Significance was to a p-value level of <0.05, corrected for multiple comparisons using FDR. Then, two multivariate analyses were performed with the significant CNV: 1) multivariate regression additive model to identify CNV independently associated with HGSC; 2) multivariate lasso regression analysis to identify which CNV predicted HGSC.

Validation was performed in two independent RNA-seq experiments. First, CNV was identified in the RNA-seq database from the UI with superFreq. The CNVs found to be significant in the univariate analysis of the DNA WES were assessed in the resulting RNA-seq CNV data. A lasso prediction model was created with these CNV resulting from the RNA-seq data, to validate the prediction model created with the significant CNV in the WES dataset. Additionally, a new multivariate lasso prediction model was created with the RNA-seq data, independent of the initial WES model, and their respective performances were compared. Finally, a multivariate logistic regression of the significant CNV from the WES data was performed in the RNA-seq data, to identify independently significant CNV.

The second validation was performed in TCGA HGSC database. Level 3, or segmentation files, resulting from TCGA HGSC dataset initial array comparative genomic hybridization experiments (CGH) were downloaded, as detailed previously. This database was chosen because it has matched normal samples for the comparison. Circular binary segmentation was used to identify regions with altered copy number in each chromosome. The copy number at a particular genomic location was computed based on the segmentation mean log-ratio data. The database was first assessed for the presence of significant CNV in the original, WES univariate analysis. With the resulting CNV a univariate analysis was performed to determine which had significant changes in their copy number (p-value <0.05), represented by their mean log-ratio. Using the genes with CNV present in the TCGA database, a new lasso prediction model was created with WES data, and then validated in this new TCGA data. Finally, a multivariate logistic analysis of all CNV present in TCGH CGH arrays and WES data was carried out to identify independently CNV associated with HGSC.

### Structural Variation (SV)

RNA-seq data from UI database (both tumor specimens and controls) was used to assess structural variation using MINTIE. RNA-seq data was used because rearrangements can be more reliably identified in the transcriptome, especially fusion transcripts. MINTIE is an integrated pipeline for RNA-seq data that takes a reference-free approach, combining de novo assembly of transcripts with differential expression analysis to identify up-regulated novel variants in a case-control setting. Counts for each SV in each sample are compared with counts in all controls. Then a comparison of normalized counts is done, a p-value is generated that is corrected with FDR (p-value <10⁻⁵). Counts between each case and control are used later for further comparison with the edgeR package. Briefly, counts are normalized, log transformed and used for univariate logistic regression. SVs with significant differential expression (p-value <0.001, to adjust for multiple comparisons) were then introduced in multivariate analyses, in an additive model first, to identify independently significant SV for HGSC, and in a lasso regression model later to identify predictors of HGSC. Significant variations were classified into type: fusion genes, alternative splicing, deletions, extended exons, intragenic rearrangement, insertion, novel exon, retained intron, novel exon, novel exon junction, and partial novel junction.

Validation was then performed in TCGA RNA-seq data and using UI RNA-seq from UI fallopian tubes for controls, because MINTIE does not need to run on matched normal tissues or controls. Initially, BAM files were converted to fastq files with bedtools for the analysis. Then, MINTIE was used to identify all SVs in TCGA dataset. SVs significant in the UI dataset that were also present in TCGA were used for validation. Briefly, significant SVs in the comparison of normalized counts for each SV in each sample versus counts in all controls (corrected p-value with FDR<10⁻⁵) were introduced in multivariate analyses, in an additive model first, to identify independently significant SV for HGSC, and in a lasso regression model later to validate UI predictors of HGSC. For this validation, only those SVs were selected that were present in both UI and TCGA databases: a) first a prediction model of HGSC with lasso regression in the UI database with these common SVs was constructed; and b) then the prediction model created in UI was validated with TCGA data.

### Results

### Single Nucleotide Variation:

The workflow for the SNV analysis is represented in FIG. 1. SNVs were identified with the VEP and superFreq methods.

a. VEP Analysis:

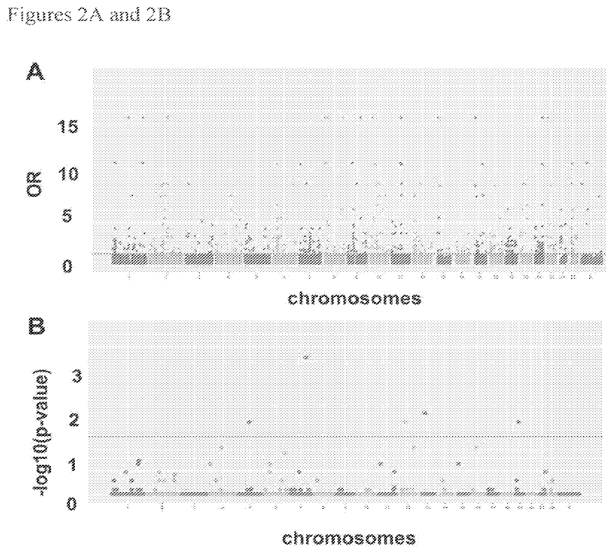

30,951,598 variants for all samples, located in 11,342,380 unique loci, were identified in our initial analysis in both the tumor and control specimens. 242,320 loci were also identified in the gnomAD database (FIG. 1) and were removed from the analysis, leaving 11,100,060 unique loci for model construction. Multiple univariate analyses of SNVs located in these 11 million loci and comparing tumor versus control samples resulted in 16,631 SNVs associated with HGSC that were selected for the multivariate analysis (p<0.001) (FIG. 2A).

b. superFreq Analysis:

220,293 SNVs within 118,839 unique loci were identified in the specimens. 100,164 of these unique loci were also present in either gnomAD database or the VEP analysis. This left 18,675 unique loci identified in the superFreq database which were not identified in the VEP analysis (FIG. 1). When comparing cases and controls for SNVs located in these loci, five were found to be below the cut-off level in multiple univariate analyses (p<0.05) (FIG. 2B).

The pathway analysis from the KEGG database was performed with 7,151 genes harboring all 16,636 SNVs identified in both analyses.

c. Prediction Modeling:

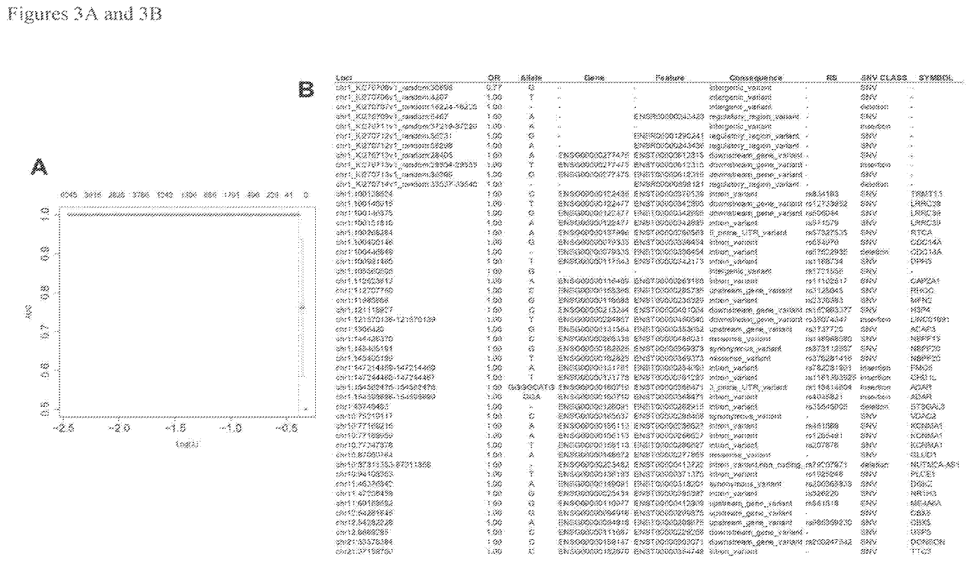

A multivariate lasso regression using selected SNVs from all univariate analyses, resulted in a model with 49 SNV, with a performance measured in AUC of 1.0 (FIGS. 3A and 3B). One of these 49 loci conferred significant protection for ovarian cancer, while the risk (OR) for HGSC of all others was minimal.

d. Validation in RNA-seq:

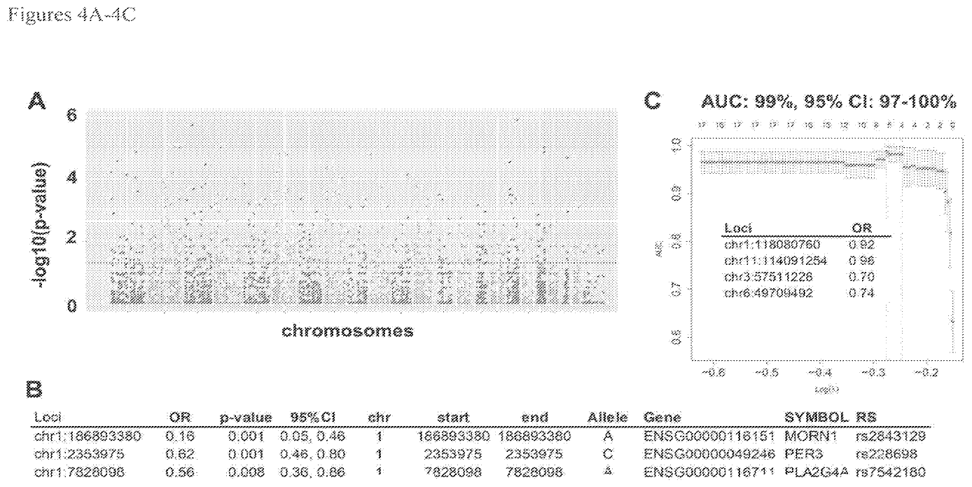

In the VEP analysis, 105,296,475 variants for all samples were identified, located within 3,276,351 unique loci in the RNA-seq data. When compared to the 11,100,060 unique loci identified in the WES data, 1,254,958 unique loci were common to both the WES and RNA-seq datasets. Of the 16,631 significant SNVs associated with HGSC from the WES analysis, 6,296 of these were also identified in the RNA-seq data and therefore common to both datasets. Using these common selected SNVs in the WES set identified in the RNA-seq analysis, multiple univariate regression were performed to determine their association with HGSC. 532 were found to be associated with HGSC (p<0.05) (FIG. 4A). In the multivariate regression analysis, including these 532 SNV significant in the univariate analysis, three of these SNVs were independently associated with HGSC, all of which were protective (FIG. 4B). A multivariate lasso regression model to predict HGSC was then created with all 532 SNV significant in the univariate analysis, resulting in a model with four SNV with an AUC of 0.99 (95% CI. 0.97-1.00) (FIG. 4C).

superFreq SNV analysis was also performed using the RNA-seq data. However, there were no significant SNV, comparing tumor and controls samples, that were present in both the WES and RNA-seq data, but not present in the VEP analysis.

Pathway enrichment analysis was performed with genes harboring the 532 significant SNVs. The most significant pathways included the Fox0 pathway and GnRH signaling pathway.

Additionally, the TCGA database was used to further validate the results. When cross-referencing the unique loci in TCGA with the 16,631 identified in the VEP analysis with WES data, 8,427 were common between the two datasets. No further comparisons were possible because of the lack of RNA-seq from controls in the HGSC TCGA set.

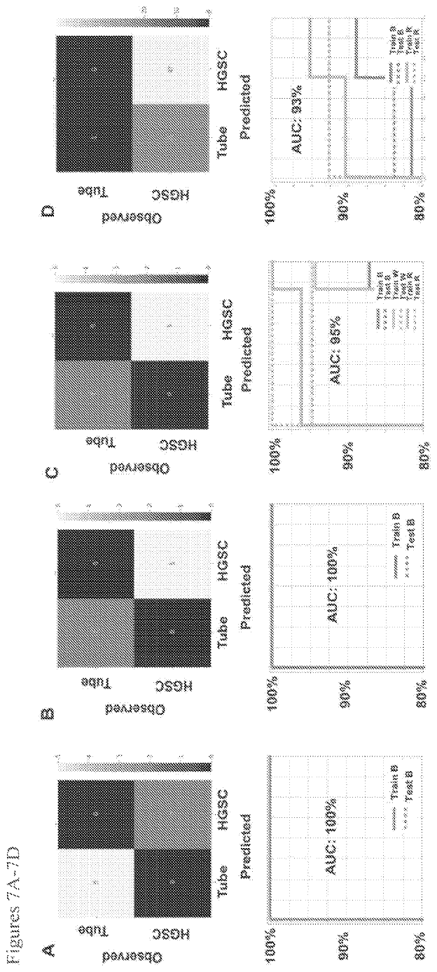

Validation of both models performed with machine learning (TensorFlow) classification with all significant SNV associated with HGSC (N=16,631) and with SNV from the multivariate lasso model (N=49) had an excellent performance, with an AUC of 1.00 (FIGS. 7A and 7B). These models performed very well without any correction. Models with RNA-seq data also performed well but lower than those with DNA SNVs (FIGS. 7C and 7D). The TensorFlow model with UI RNA-seq data, with 20 SNVs present in both DNA and RNA, had and AUC of 0.95, after accounting for unbalanced data (tubes represented less than 10% of the total samples). TCGA RNA-seq model, with 18 SNV present (out of the initial 49 SNVs from DNA) had an AUC of 0.93, also after accounting for unbalanced data (we used UI controls to construct the model).

### Copy Number Variation (CNV):

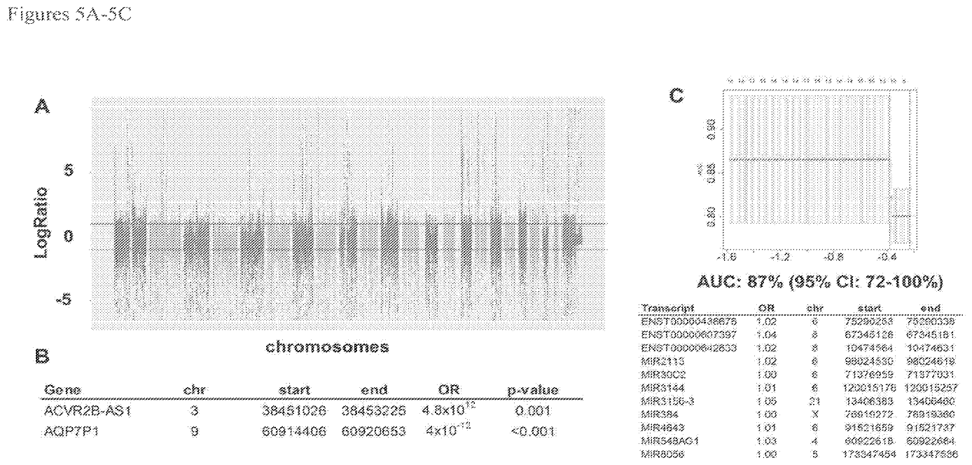

CNV assessed using superFreq in the DNA WES identified 558 genes (out of the 23,443 genes assessed) that had different CNV between HGSC and controls (FIG. 5A). The multivariate logistic regression determined that two of these CNV were independently associated with HGSC (FIG. 5B). A multivariate lasso regression including all 558 significant CNV, resulted in a prediction model with 11 CNV that would predict HGSC with a performance by AUC of 0.87 (95% CI: 0.72-1.00; FIG. 5C). Pathway enrichment analysis including all significant CNVs yielded one significant pathway, Herpes Simplex Virus 1 infection.

Validation of CNV results using RNA-seq data from UI specimens also was processed with superFreq. The 558 transcripts identified in the WES analysis were assessed. The validation of the CNV prediction model for HGSC had an AUC of 0.67 when using RNA-seq data. A lasso prediction model, independent of the DNA model, and constructed with the 588 significant CNVs with the RNA-seq data resulted in a prediction model of HGSC with two CNVs and an AUC of 1.00 (95% CI: 1.00-1.00). One gene significant in the multivariate analysis increased the risk for cancer (KRT14) and the other decreased the risk for HGSC (DENND1C).

In TCGA CGH database, 162 of the 588 CNVs significantly different between cases and controls in the WES analysis were present. 148 of these 162 were found to be significantly associated with HGSC as compared to normal controls. When a new lasso regression analysis was done in the WES CNV dataset using only CNV that were present in TCGA (N=162) the resulting prediction model with six CNV had an inferior performance, with an AUC of 0.8 (95% CI: 0.74-0.86). When this model was validated in TCGA data the performance of the model had an AUC of 50%.

### Structural Variation (SV):

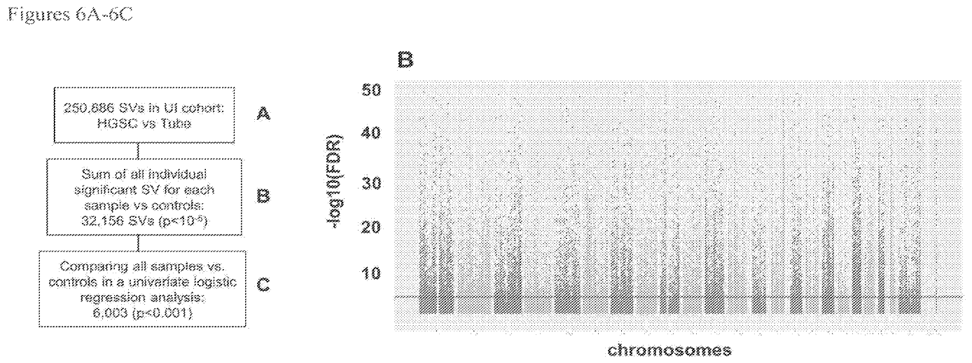

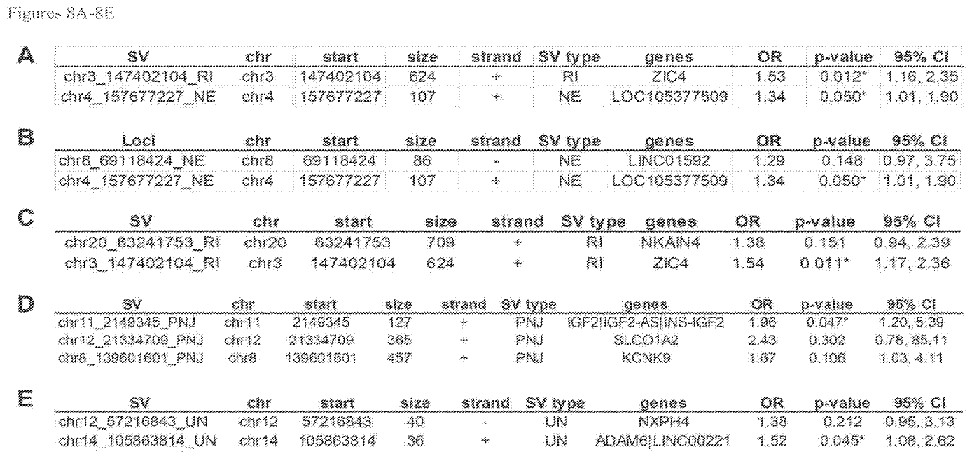

In total, there were 250,886 SV of all types detected in the UI RNA-seq when comparing HGSC with normal tubes samples (FIG. 6A). When comparing counts for each SV in each sample with all controls, 32,156 were significant (p-value <10⁻⁵) (FIG. 6B). Multiple univariate logistic regressions were performed to select 6,003 of these SV associated with HGSC for the multivariate model (FIG. 6C). 75 of these SV were decreased in HGSC and 5,928 were increased. Multivariate analyses for all structural variations collectively as well as for each type of structural variation are reported in FIG. 8A-8E.

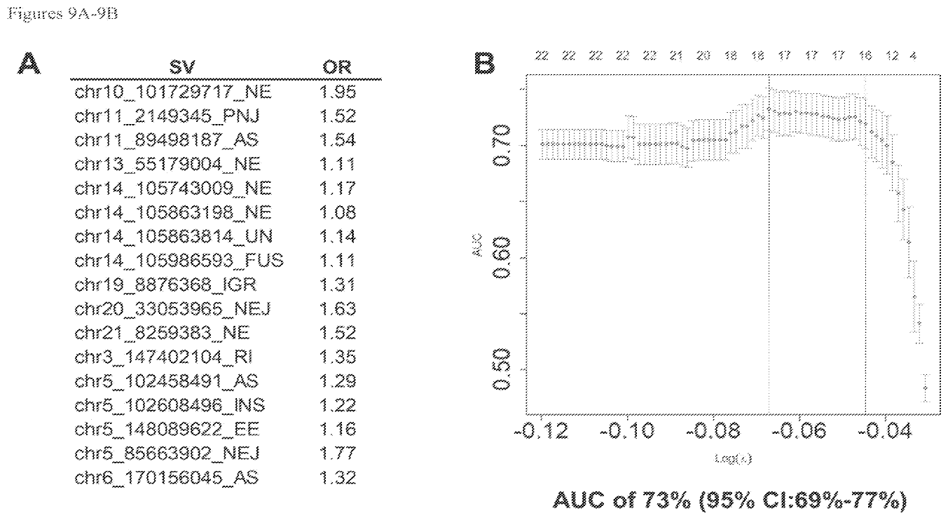

A multivariate lasso regression analysis identified 17 SV model that predicted HGSC with an AUC of 0.73 (95% CI: 0.69-0.77) (FIGS. 9A-9B). TCGA validation detected a total of 101,567 SVs in HGSC RNA-seq samples versus all controls. Out of the 6,003 SVs significantly associated with HGSC in the UI analysis, 3,429 were present in TCGA set, and 3,353 of these were significant when comparing counts for each SV in each sample with all controls (p<10⁻⁵). A multivariate logistic regression analysis of TCGA significant SVs showed 2 SV independently associated with HGSC. A new lasso regression analysis was performed with UI data using only those 3,429 SVs present in TCGA. It resulted in a prediction model with an AUC of 0.71 (95% CI: 0.63-0.79). Validation of this model with TCGA data performed very similar to the UI model with an AUC of 0.74 (95% CI: 0.71-0.77).

### Discussion

The overarching goal of this study was to identify genetic/genomic variation that could differentiate HGSC from normal tubal tissue. This could lead to the discovery of methods that finally will be able to detect HGSC early and by non-invasive means. With that aim, DNA and RNA sequencing was used to identify a comprehensive array of genetic/genomic variation in HGSC and benign fallopian tubes. Then, those DNA and RNA characteristics were used to create prediction models that would discriminate accurately and robustly, by validating those models in different settings, in independent databases, in different platforms, and with different analytics. The SNV model carried the highest accuracy with an AUC of 1.00 when using 49 different SNV loci, while the models for copy number variation and structural variation were successful, but somewhat less accurate with AUC of 0.87 and 0.73, respectively. These models were then successfully validated using RNA-seq data from our dataset as well as TCGA, and the SNV data was even further validated using machine learning with TensorFlow. Initially, the SNV model is the most promising, but those created for CNV and SV also had merit.

It is well known that circulating tumor DNA (ctDNA) and RNA can be isolated from peripheral blood draws in patients. Circulating tumor DNA is identifiable in ovarian cancer patient plasma with reliable accuracy, and SNV, CV, and SV have all been identified in peripheral specimens. Using this knowledge and the models created herein, it is feasible to identify the variations used in the models through a peripheral blood draw on patients with pelvic masses of unknown etiology. ctDNA was then amplified and the necessary variants identified using a pre-defined panel. ctDNA is a better medium for this type of testing because it is more stable in an extracellular form than RNA, though circulating tumor RNA has also been identified and sequenced. This constitutes a method to diagnose ovarian cancer with a “liquid biopsy.”

A clinical use for these prediction models is for early diagnosis and screening; a method which could significantly improve patient survival if we could increase the percentage of patients diagnosed at Stage I and II. ctDNA has been identified in ovarian cancer patients at early stages. Panels for other cancers have been created to drive individualized treatment, cancer surveillance, and early diagnosis; two of which have recently gained FDA approval. Given that ovarian cancer ctDNA is identifiable in patients with early-stage ovarian cancer, and that the model predicts ovarian cancer with high accuracy, the model described herein has the potential to provide a strong diagnostic tool to diagnose ovarian cancer at an early stage.

Additionally, this method could be used to identify recurrent or persistent cancer who have completed their adjuvant therapy. Liquid biopsy has already been shown to be feasible in identifying recurrence in ovarian cancer and these methods are currently being investigated in the monitoring of lung cancer. While these would likely require different models to identify those variations which are present in recurrent cancer, the methods for creating them would be the same.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A panel of probes that hybridize to nucleic acid, the panel comprising at least 18 probes selected from the group consisting of probes listed in FIG. 3B.

2. The panel of probes of claim 1, wherein the panel comprises at least 40 probes.

3. The panel of probes of claim 1, wherein the panel comprises at least 49 probes.

4. The panel of probes of claim 1, wherein the probes are specific for single nucleotide variants (SNVs).

5. The panel of probes of claim 1, wherein the probes are specific for copy number variants (CNVs).

6. The panel of probes of claim 1, wherein the probes are specific for structural variants (SVs).

7. The panel of claim 1, wherein each probe comprises a unique label.

8. A kit comprising the panel of probes of claim 1 and instructions for use in analyzing a biological sample.

9. The kit of claim 21, wherein the biological sample is a liquid biopsy.

10. The kit of claim 22, wherein the liquid biopsy is blood or a blood product.

11. A method of detecting the presence of biomarkers associated with an increased risk of ovarian cancer in a human subject, comprising:
(i) contacting DNA from a biological sample containing cells from the subject with the panel of probes of any one of claims 1-6, wherein the panel comprises probes specific for single nucleotide variants (SNVs) to form hybridized target sequences;
(ii) detecting the hybridized target sequences;
(iii) determining the number of hybridized target sequences detected; and
(iv) indicating that the human subject has an increased risk of ovarian cancer if more than 18 SNVs are present.

12. The method of claim 11, wherein the biological sample is a liquid biopsy.

13. The method of claim 12, wherein the liquid biopsy is blood or a blood product.

14. The method of any one of claims 11-13, wherein the biological sample is subdivided into individual subsamples, and a different single probe is applied to each subsample.

15. A method of treating a human subject for ovarian cancer, comprising:
(i) collecting a liquid biopsy from the subject, and extracting ctDNA from the liquid biopsy;
(ii) analyzing the presence or absence of ctDNA listing in a panel of loci selected from the group consisting of probes listed in FIG. 3B;
(iii) determining the number of matching loci are present;
(iv) indicating that the human subject has an increased risk of ovarian cancer if at least 18 loci are present; and
(v) administering an appropriate treatment to the patient.

16. The method of claim 15, wherein the panel comprises at least 40 probes.

17. The method of claim 15, wherein the panel comprises at least 49 probes.

18. The method of any one of claims 15-17, wherein the treatment comprises assessment of fallopian tubes and ovaries with imaging techniques or removal of fallopian tubes and ovaries.

19. A method of determining an increased risk or presence of ovarian cancer in a human subject comprising:
(i) collecting a liquid biopsy from the subject, and extracting ctDNA from the liquid biopsy;
(ii) analyzing the presence or absence of ctDNA listing in a panel of loci selected from the group consisting of probes listed in FIG. 3B;
(iii) determining the number of matching loci are present; and
(iv) indicating that the human subject has an increased risk of ovarian cancer if at least 18 loci are present.

20. The method of claim 19, wherein the panel comprises at least 40 probes.

21. The method of claim 19, wherein the panel comprises at least 49 probes.
